(19) United States
(12) Patent Application Publication
(10) Pub. No.: US 2012/0122107 A1
(43) Pub. Date: May 17, 2012

(54) HOMEOSTATIC MULTIDOMAIN PROTEIN, AND USES FOR IT

(75) Inventors: Steffen Thiel, Risskov (DK); Søren Degn, Viby J (DK); Jens Christian Jensenius, Odense M (DK)

(73) Assignee: AARHUS UNIVERSITET, ARHUS C (DK)

(21) Appl. No.: 12/947,751

(22) Filed: Nov. 16, 2010

(51) Int. Cl. C12Q 1/68; G01N 33/566; C07H 21/04; C12N 15/63; C07K 14/00; C12N 15/12

(52) U.S. Cl. 435/6.12; 530/350; 530/324; 536/23.5; 536/23.1; 435/320.1; 436/501

(57) ABSTRACT

The invention relates to the discovery and characterization of mannan binding lectin-associated protein (map44), a new protein that acts in the lectin pathway of complement activation.

### FIELD OF THE INVENTION

The invention is in the general field of innate immune defense, inflammation and the pathways for complement fixation involving the proteins mannan-binding lectin (MBL), also termed mannan-binding protein or mannose-binding protein (MBP) and ficolins (including H-ficolin, L-ficolin and M-ficolin, also termed ficolin-3, -2, and -1, respectively).

### BACKGROUND OF THE INVENTION

The recognition molecules of the innate immune system include the soluble pattern recognition molecules (sPRMs) with collagen-like regions: mannan-binding lectin (MBL) and the three ficolins (H-, L- and M-ficolin), collectively termed collagenous lectins or “collectins”. Upon recognition of patterns of ligands they initiate the complement cascade through activation of proenzymes, MBL-associated serine proteases (MASPs) (Thiel et al., Complement activating soluble pattern recognition molecules with collagen-like regions, mannan-binding lectin, ficolins and associated proteins. Mol. Immunol., 2007; 44(16):3875-88). The complement system plays a central role in the innate immune system. Upon activation it facilitates direct microbial killing but also acts as a natural adjuvant, enhancing and directing the adaptive immune response (Dempsey et al., 1996, C3d of complement as a molecular adjuvant: Bridging innate and acquired immunity. Science 271, 348-350). Three different pathways that may lead to activation of the complement system have been described: the classical pathway initiated by antibody-antigen complexes, the alternative pathway initiated by certain structures on microbial surfaces and the lectin pathway of complement activation (Volanakis, J. E. & Frank, M. M. (eds.) The Human Complement System in Health and Disease. Marcel Decker Inc., New York (1998). The later pathway is initiated when mannan-binding lectin (MBL, first described as mannan-binding protein, MBP, see Ezekowitz, U.S. Pat. No. 5,270,199) binds to carbohydrates or when ficolins bind to a suitable target (Thiel, supra). The targets for MBL and ficolins include surface structures on a range of microorganisms such as bacteria, yeast, parasitic protozoa and viruses.

The homologous proteases MASP-1 and -3 are encoded by the MASP1 gene (Matsushita, M. & Fujita, T., Activation of the classical complement pathway by mannose-binding protein in association with a novel C1s-like serine protease J. Exp. Med. 176, 1497-1502 (1992) and Dahl M R et al, MASP-3 and its association with distinct complexes of the mannan-binding, lectin complement activation pathway. Immunity. 2001 July; 15(1):127-35), while MASP-2 and a short alternative splice product, MAp19, are encoded by the MASP2 gene (Thiel et al., A second serine protease associated with mannan-binding lectin that activates complement. Nature. 1997 Apr. 3; 386(6624):506-10 and Schwaeble et al., The mannan-binding lectin-associated serine proteases (MASPs) and MAp19: four components of the lectin pathway activation complex encoded by two genes. Immunobiology. 2002 September; 205(4-5):455-66). The three MASPs and MAp19 form homodimers, which associate with MBL and ficolins through their N-terminal domains. Activated MASP-2 cleaves the complement factors C4 and C2 to generate C3 convertase (Thiel, 2007, supra and Thiel et al., 1997, supra). The functions of MASP-1, MASP-3 and MAp19 remain unresolved, although MASP-1 has been shown to cleave C2 with significant activity (Matsushita et al., Proteolytic activities of two types of mannose-binding lectin-associated serine protease. J Immunol. 2000 Sep. 1; 165(5):2637-42), leading to the suggestion that MASP-1 cooperates with MASP-2 in generating C3 convertase (Møller-Kristensen et al., Cooperation between MASP-1 and MASP-2 in the generation of C3 convertase through the MBL pathway. Int Immunol. 2007 February; 19(2):141-9). MASP-1 has also been suggested to cleave protease activated receptors (PARs) (Megyeri et al., Complement protease MASP-1 activates human endothelial cells: PAR4 activation is a link between complement and endothelial function. J Immunol. 2009 Sep. 1; 183(5):3409-16) and complement factor D thus influencing fat metabolism (Takahashi et al., Contributions of MASP-1 and MASP-3 to fat metabolism by activation of complement factor D, Molecular Immunology, 46 (14), 2009, Page 2828).

This pathway of complement activation is clinically relevant as, e.g., genetically determined MBL deficiency is associated with susceptibility to frequent infections with a variety of microorganisms in childhood, and in adults (Dommett et al., Mannose-binding lectin in innate immunity: past, present and future, Tissue Antigens. 2006 September; 68(3):193-209), especially when the immune defence is otherwise compromised, such as during treatment for cancer or due to infection with HIV, where MBL deficiency is also associated with more rapid death following development of AIDS. MBL deficiency is also associated with a predisposition to recurrent spontaneous abortions, and also to development of systemic lupus erythrematosus. In the first clinical reconstitution trial, an infant MBL-deficient girl suffering from recurrent infections was apparently cured by injections with purified MBL. For a recent reviews on MBL, see, e.g., Dommett et al., supra and Mette Møller-Kristensen et al., 2009, MBL polymorphisms and infectious diseases, 303-332, in “Animal lectins: A functional view”, eds. Vasta and Ahmed, CRC Press, Taylor and Francis Group).

Relatively high frequencies of MBL mutations associated with MBL-deficiency have been reported in all populations studied. This observation has led to the hypothesis that MBL may, in certain cases, render the individual more susceptible to certain intracellular infectious agents exploiting MBL to gain access to the target tissues. Since MBL is a very powerful activator of the complement system, inexpedient activation through microbial carbohydrates or endotoxins can lead to damaging inflammatory responses.

### SUMMARY OF THE INVENTION

The invention relates to the isolation and characterization of a collectin-associated protein (MAP44). MAP44 shows homology with parts of the previously reported MASPs (MASP-1, MASP-2 and MASP-3).

We have purified MAP44 and characterized it by its association with collectin, its molecular size and its partial amino acid sequence. We have cloned a cDNA fragment and determined its base sequence, which translates into an amino acid sequence encompassing some of the sequenced peptides. Like MASP-1, MASP-2 and MASP-3, MAp44 co-purifies with MBL, and is likely to be involved in the biological effects of the MBL complex as well as ficolin complexes.

Thus, one aspect of the invention features substantially pure MAp44 polypeptides and nucleic acids encoding such polypeptides. Preferably, the MAP44 polypeptide retains one or more MAP44 functions, such as being capable of associating with mannan-binding lectin (MBL) or ficolins, a substantially pure MAP44 polypeptide, preferably a polypeptide being capable of associating with mannan-binding lectin (MBL) or ficolins.

Another aspect is the production of anti-MAP44 antibodies and the use of such antibodies for the construction of assays for MAP44 and the use of such assays for determining clinical syndromes associated with over- or under-expression of this protein, such as an antibody produced by administering an MAP44 polypeptide, or part of the MAP44 polypeptide, or DNA encoding any such polypeptide, according to claim 1 to an animal with the aim of producing antibody.

Some MAP44 polypeptides according to the invention, e.g., those used in binding assays, may be conjugated to a label so as to permit detection and/or quantification of their presence in the assay. Suitable labels include enzymes, which generate a signal (e.g., visible absorption), fluorophores, radionuclides, etc. MAP44 polypeptides are capable of competitively inhibiting one of the MASP-1, MASP-2 or MASP-3 activities, and thereby are useful in evaluating MAP44 function. Other MAP44 poly-peptides are useful antigens or haptens for producing antibodies as described below. Compounds, which competitively inhibit a MAP44 activity, are also featured. Preferably, such compounds act by inhibiting the activity of MAP44 or of a fragment of MAP44. Such compounds may include fragments of MBL or ficolins or of MAP44 or MASP-1 or MASP-3, which competitively inhibit the MBL-MAP44 or ficolin-MAP44 interactions critical to the function of the complex.

Specific polypeptides according to this aspect of the invention include: a) a polypeptide with a molecular mass of approximately 44 kDa and containing or comprising the sequence identified as SEQ ID NO:1 including any functional equivalent thereof.

Another aspect of the invention includes an isolated nucleic acid molecule comprising a nucleotide sequence encoding a polypeptide encompassing sequences that are at least 85% identical, such as at least 90% identical, for example at least 95% identical to any of the sequences of SEQ ID NO:1 and the coding part of SEQ ID NO:2, i.e., the part of the sequence starting with nucleotide no. 333 (a), and ending with nucleotide no. 1475.

Thus, the invention relates to an isolated nucleic acid molecule encoding the polypeptide according to the invention, the molecule comprising a nucleotide sequence encoding a polypeptide having sequence that is at least 50% identical to the sequence of SEQ ID NO:1.

The invention also features isolated nucleic acid sequences encoding the above mentioned MAP44 polypeptides. Such nucleic acid sequences may be included in nucleic acid vectors (e.g., expression vectors including those with regulatory nucleic acid elements permitting expression of recombinant nucleic acid in an expression system).

The invention also features isolated nucleic acid sequences encoding polypeptides of the entire 44 kDa MAP44 protein. Such nucleic acid sequences may be included in nucleic acid vectors (e.g., expression vectors including those with regulatory nucleic acid elements permitting expression of recombinant nucleic acid in an expression system).

The invention also features antibodies that selectively bind to MAP44. Such antibodies may be made by any of the well-known techniques including polyclonal and monoclonal antibody techniques. The antibody may be coupled to a compound comprising a detectable marker, so that it can be used, e.g., in an assay to detect MAP44.

The polypeptides or antibodies may be formulated into pharmaceutical compositions and administered as therapeutics as described below.

The invention also features methods for detecting MAP44. The method comprises; obtaining a biological sample, contacting the biological sample with a MAP44 polypeptide specific binding partner, and detecting the bound complexes, if any, as an indication of the presence of MAP44 in the biological sample. The binding partner used in the assay may be an antibody, or the assay for MAP44 may test for complement fixing activity. These assays for MAP44 may also be used for quantitative assays of MAP44 or MAP44 activity in biological samples. One of the binding partners may be specific for MBL or ficolins thus allowing for the detection of MBL/MAP44 or ficolin/MAP44 complexes.

Methods for detecting MAP44 nucleic acid expression are included in the invention. These methods comprise detecting RNA having a sequence encoding a MAP44 polypeptide by mixing the sample with a nucleic acid probe that specifically hybridizes under stringent conditions to a nucleic acid sequence encoding all or a fragment of MAP44.

The invention also features methods for treating patients deficient in MAP44 or MAP44 activity. This is accomplished by administering to the patient MAP44 polypeptide or nucleic acid encoding MAP44. Because it is sometimes desirable to inhibit MAP44 activity, the invention includes a method for inhibiting the activity of MAP44 by administering to the patient a compound that inhibits expression or activity of MAP44. Inhibition of MAP44 activity may also be achieved by administering a MAP44 anti-sense nucleic acid sequence.

The invention features an assay for polymorphisms in the nucleic acid sequence encoding MAP44. A method of detecting the presence of MAP44-encoding nucleic acid in a sample is claimed. As an example, the method may include mixing the sample with at least one nucleic acid probe capable of forming a complex with MAP44-encoding nucleic acid under stringent conditions, and determining whether the probe is bound to sample nucleic acid. The invention thus includes nucleic acid probe capable of forming a complex with MAP44-encoding nucleic acid under strin-gent conditions.

The invention features an assay for polymorphisms in the polypeptide sequence comprising MAP44 or its precursor or MAP44 regulatory sequences.

MAP44 assays are useful for the determination of MAP44 levels and MAP44 activity in patients suffering from various diseases such as infections, inflammatory diseases and spontaneous recurrent abortion. MAP44 is useful for the treatment of infections when MAP44 function is suboptimal, and inhibition of MASP activity is useful for regulation of inflammation and adverse effects caused by activity of MAP44.

Furthermore, the invention relates to the use of a polypeptide as defined herein for preparation of a pharmaceutical composition.

By MAP44 is meant the polypeptide or any other polypeptide having substantial sequence identity with SEQ ID NO:1.

The terms “protein” and “polypeptide” are used herein to describe any chain of amino acids, regardless of length or post-translational modification (for example, glycosylation or phosphorylation). Thus, the term “MAP44 polypeptide” includes full-length, naturally occurring MAP44 protein, as well as recombinantly or synthetically produced polypeptide that corresponds to a full-length naturally occurring MAP44 polypeptide, or to particular domains or portions of a naturally occurring protein. The term also encompasses mature MAP44 which has an added amino-terminal methionine (which is useful for expression in prokaryotic cells).

The term “purified” as used herein refers to a nucleic acid or peptide that is substantially free of cellular material, viral material, or culture medium when produced by recombinant DNA techniques, or chemical precursors or other chemicals when chemically synthesized.

By “isolated nucleic acid molecule” is meant a nucleic acid molecule that is separated in any way from sequences in the naturally occurring genome of an organism. Thus, the term “isolated nucleic acid molecule” includes nucleic acid molecules, which are not naturally occurring, e.g., nucleic acid molecules created by recombinant DNA techniques.

The term “nucleic acid molecule” encompasses both RNA and DNA, including cDNA, genomic DNA, and synthetic (e.g., chemically synthesized) DNA. Where single-stranded, the nucleic acid may be a sense strand or an antisense strand.

The invention also encompasses nucleic acid molecules that hybridize, preferably under stringent conditions, to a nucleic acid molecule encoding an MAP44 polypeptide or any other part of the entire cDNA encoding the complete MAP44 sequence. In addition, the invention encompasses nucleic acid molecules that hybridize, preferably under stringent conditions, to a nucleic acid molecule having the sequence of the MAP44 encoding cDNA contained in a clone. Preferably the hybridizing nucleic acid molecule consists of 400, more preferably 200 nucleotides.

Preferred hybridizing nucleic acid molecules encode an activity possessed by MAP44, e.g., they bind MBL or ficolins (or another MAP44 ligand).

The invention also features substantially pure or isolated MAP44 polypeptides, preferably those that correspond to various functional domains of MAP44, or fragments thereof. The polypeptides of the invention encompass amino acid sequences that are substantially identical to parts of the amino acid sequence shown in SEQ ID NO:1, or substantially identical to the amino acid sequence of the entire MAP44 protein.

The polypeptides of the invention can also be chemically synthesized, synthesized by recombinant technology, or they can be purified from tissues in which they are naturally expressed, according to standard biochemical methods of purification.

Also included in the invention are “functional polypeptides” which possess one or more of the biological functions or activities of MAP44. These functions or activities are described in detail in the specification. A functional polypeptide is also considered within the scope of the invention if it serves as an antigen for production of antibodies that specifically bind to MAP44 or fragments (particularly determinant containing fragments) thereof.

The functional polypeptides may contain a primary amino acid sequence that has been modified from those disclosed herein. Preferably these modifications consist of conservative amino acid substitutions, as described herein. The polypeptides may be substituted in any manner designed to promote or delay their catabolism (increase their half-life).

Conservative amino acid substitutions as used herein relate to the substitution of one amino acid (within a predetermined group of amino acids) for another amino acid (within the same group) exhibiting similar or substantially similar characteristics.

Within the meaning of the term “conservative amino acid substitution” as applied herein, one amino acid may be substituted for another within groups of amino acids characterised by having

i) polar side chains (Asp, Glu, Lys, Arg, His, Asn, Gln, Ser, Thr, Tyr, and Cys,)

ii) non-polar side chains (Gly, Ala, Val, Leu, Ile, Phe, Trp, Pro, and Met)

iii) aliphatic side chains (Gly, Ala Val, Leu, Ile)

iv) cyclic side chains (Phe, Tyr, Trp, His, Pro)

v) aromatic side chains (Phe, Tyr, Trp)

vi) acidic side chains (Asp, Glu)

vii) basic side chains (Lys, Arg, His)

viii) amide side chains (Asn, Gln)

ix) hydroxy side chains (Ser, Thr)

x) sulfur-containing side chains (Cys, Met), and

xi) amino acids being monoamino-dicarboxylic acids or monoamino-monocarboxylic-monoamidocarboxylic acids (Asp, Glu, Asn, Gln).

When the amino acid sequence comprises a substitution of one amino acid for another, such a substitution may be a conservative amino acid substitution as defined herein above. Fragments of MAP44 according to the present invention may comprise more than one such substitution, such as, e.g., two conservative amino acid substitutions, for example three or four conservative amino acid substitutions, such as five or six conservative amino acid substitutions, for example seven or eight conservative amino acid substitutions, such as from 10 to 15 conservative amino acid substitutions, for example from 15 to 25 conservative amino acid substitutions. Substitutions can be made within any one or more groups of predetermined amino acids as listed herein above.

The addition or deletion of an amino acid may be an addition or deletion of from 2 to preferably 10 amino acids, such as from 2 to 8 amino acids, for example from 2 to 6 amino acids, such as from 2 to 4 amino acids. However, additions of more than 10 amino acids, such as additions from 10 to 200 amino acids, are also comprised within the present invention.

It will thus be understood that the invention also pertains to immunogenic composition comprising at least one fragment of MAP44, including any variants and functional equivalents of such at least one fragment.

The fragment of MAP44 according to the present invention, including any variants and functional equivalents thereof, may in one embodiment comprise less than 100 amino acid residues, such as less than 95 amino acid residues, for example less than 90 amino acid residues, such as less than 85 amino acid residues, for example less than 80 amino acid residues, such as less than 75 amino acid residues, for example less than 70 amino acid residues, such as less than 65 amino acid residues, for example less than 60 amino acid residues, such as less than 55 amino acid residues, for example less than 50 amino acid residues.

Functional equivalency as used in the present invention is according to one preferred embodiment established by means of reference to the corresponding functionality of a predetermined MAP44 fragment, such as, e.g., the fragment comprising or essentially consisting of the first two domains, the first three domains or the first four domains of MAP44, or a full length MAP44 sequence.

Functional equivalents of a fragment of MAP44 comprising a predetermined amino acid sequence is defined as stated herein above. One method of determining a sequence of immunogenically active amino acids within a known amino acid sequence has been described by Geysen in U.S. Pat. No. 5,595,915 and is incorporated herein by reference.

A further suitably adaptable method for determining structure and function relationships of peptide fragments is described by U.S. Pat. No. 6,013,478, which is herein incorporated by reference.

Functional equivalents of fragments of MAP44 will be understood to exhibit amino acid sequences gradually departing from the preferred predetermined sequence including a sequence comprising or essentially consisting of a MAP44 B-chain, as the number and scope of insertions, deletions and substitutions including conservative substitutions increases. This departure is measured as a reduction in homology between the preferred predetermined sequence and the variant or functional equivalent.

All MAP44 fragments that are active as inhibitors are included within the scope of this invention, regardless of the degree of homology that they show to a preferred predetermined sequence of MAP44. The reason for this is that some regions of MAP44 are most likely readily mutatable, or capable of being completely deleted, without any significant biological effect.

A functional variant obtained by substitution may well exhibit some form or degree of native MAP44 activity, and yet be less homologous, if residues containing functionally similar amino acid side chains are substituted. Functionally similar in this respect refers to dominant characteristics of the side chains such as hydrophobic, basic, neutral or acidic, or the presence or absence of steric bulk. Accordingly, in one embodiment of the invention, the degree of identity between i) a given MAP44 fragment capable of eliciting a complement stimulating or inhibitory effect and ii) a preferred predetermined fragment of MAP44, is not a principal measure of the fragment as a variant or functional equivalent of a preferred, predetermined MAP44 fragment according to the present invention.

A non-conservative substitution leading to the formation of a functionally equivalent fragment of MAP44 would for example i) differ substantially in hydrophobicity, for example a hydrophobic residue (Val, Ile, Leu, Phe or Met) substituted for a hydrophilic residue such as Arg, Lys, Trp or Asn, or a hydrophilic residue such as Thr, Ser, His, Gln, Asn, Lys, Asp, Glu or Trp substituted for a hydrophobic residue; and/or ii) differ substantially in its effect on polypeptide backbone orientation such as substitution of or for Pro or Gly by another residue; and/or iii) differ substantially in electric charge, for example substitution of a negatively charged residue such as Glu or Asp for a positively charged residue such as Lys, His or Arg (and vice versa); and/or iv) differ substantially in steric bulk, for example substitution of a bulky residue such as His, Trp, Phe or Tyr for one having a minor side chain, e.g. Ala, Gly or Ser (and vice versa).

In a further embodiment the present invention relates to functional equivalents of a preferred predetermined fragment of MAP44, wherein such equivalents comprise substituted amino acids having hydrophilic or hydropathic indices that are within +/−2.5, for example within +/−2.3, such as within +/−2.1, for example within +/−2.0, such as within +/−1.8, for example within +/−1.6, such as within +/−1.5, for example within +/−1.4, such as within +/−1.3 for example within +/−1.2, such as within +/−1.1, for example within +/−1.0, such as within +/−0.9, for example within +/−0.8, such as within +/−0.7, for example within +/−0.6, such as within +/−0.5, for example within +/−0.4, such as within +/−0.3, for example within +/−0.25, such as within +/−0.2 of the value of the amino acid it has substituted.

The importance of the hydrophilic and hydropathic amino acid indices in conferring interactive biologic function on a protein is well understood in the art (Kyte & Doolittle, 1982 and Hopp, U.S. Pat. No. 4,554,101, each incorporated herein by refer-ence).

The amino acid hydropathic index values as used herein are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5) (Kyte & Doolittle, 1982).

The amino acid hydrophilicity values are: arginine (+3.0); lysine (+3.0); aspartate (+3.0.+−.1); glutamate (+3.0.+−.1); serine (+0.3); asparagine (+0.2); glutamine (+0.2); glycine (0); threonine (−0.4); proline (−0.5.+−.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4) (U.S. Pat. No. 4,554,101).

Substitution of amino acids can therefore in one embodiment be made based upon their hydrophobicity and hydrophilicity values and the relative similarity of the amino acid side-chain substituents, including charge, size, and the like. Exemplary amino acid substitutions which take various of the foregoing characteristics into consideration are well known to those of skill in the art and include: arginine and lysine; glutamate and aspartate; serine and threonine; glutamine and asparagine; and valine, leucine and isoleucine.

In addition to the peptidyl compounds described herein, sterically similar compounds may be formulated to mimic the key portions of the peptide structure and that such compounds may also be used in the same manner as the peptides of the invention. This may be achieved by techniques of modelling and chemical designing known to those of skill in the art. For example, esterification and other alkylations may be employed to modify the amino terminus of, e.g., a di-arginine peptide backbone, to mimic a tetra peptide structure. It will be understood that all such sterically similar constructs fall within the scope of the present invention.

Peptides with N-terminal alkylations and C-terminal esterifications are also encompassed within the present invention. Functional equivalents also comprise glycosylated and covalent or aggregative conjugates formed with the same or other MAP44 fragments and/or MAP44 molecules, including dimers or unrelated chemical moieties. Such functional equivalents are prepared by in vivo synthesis or by linkage of functionalities to groups which are found in fragment including at any one or both of the N- and C-termini, by means known in the art.

Oligomers of MAP44 including dimers including homodimers and heterodimers of fragments of MAP44 according to the invention are also provided for within the scope of the present invention. MAP44 functional equivalents and variants can be produced as homodimers or heterodimers with other amino acid sequences or with native MAP44 sequences.

The terms functional MAP44 equivalents, MAP44 variants and MAP44 derivatives as used herein relate to functional equivalents of a fragment of MAP44 comprising a predetermined amino acid sequence, and such equivalents, derivatives and variants are defined as:

i) MAP44 or fragments thereof comprising an amino acid sequence capable of being recognised by an antibody also capable of recognising the predetermined amino acid sequence, and/or

ii) MAP44 or fragments thereof comprising an amino acid sequence capable of forming an association with a component of the MBL or ficolin pathway, such as the MBL/MASP-2, ficolin/MASP-2, MBL/MASP-1, ficolin/MASP-1, MBL/MASP-3 or ficolin/MASP-3 complexes, wherein said component is also capable of forming an association with the predetermined amino acid se-quence, and/or

iii) Fragments of MAP44 having at least a substantially similar complement inhibiting effect as the fragment of MAP44 comprising said predetermined amino acid sequence, such as inhibiting cleavage of C4 or protease acti-vated receptors (PARs).

Polypeptides or other compounds of interest are said to be “substantially pure” when they are distinct from any naturally occurring composition, and suitable for at least one of the uses proposed herein. While preparations that are only slightly altered with respect to naturally occurring substances may be somewhat useful, more typically, the preparations are at least 10% by weight (dry weight) the compound of interest. Preferably, the preparation is at least 60%, more preferably at least 75%, and most preferably at least 90%, by weight the compound of interest. Purity can be measured by any appropriate standard method, for example, by column chromatography, polyacrylamide gel electrophoresis, or HPLC analysis.

A polypeptide or nucleic acid molecule is “substantially identical” to a reference polypeptide or nucleic acid molecule if it has a sequence that is at least 85%, preferably at least 90%, and more preferably at least 95%, 98%, or 99% identical to the sequence of the reference polypeptide or nucleic acid molecule.

Where a particular polypeptide is said to have a specific percent identity to a reference polypeptide of a defined length, the percent identity is relative to the reference peptide. Thus, a peptide that is 50% identical to a reference polypeptide that is 100 amino acids long can be a 50 amino acid polypeptide that is completely identical to a 50 amino acid long portion of the reference polypeptide. It might also be a 100 amino acid long polypeptide which is 50% identical to the reference polypeptide over its entire length. Of course, many other polypeptides will meet the same criteria.

In the case of polypeptide sequences which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine.

For polypeptides, the length of the reference polypeptide sequence will generally be at least 16 amino acids, preferably at least 20 amino acids, more preferably at least 25 amino acids, and most preferably 35 amino acids, 50 amino acids, or 100 amino acids. For nucleic acids, the length of the reference nucleic acid sequence will generally be at least 50 nucleotides, preferably at least 60 nucleotides, more preferably at least 75 nucleotides, and most preferably 100 nucleotides or 300 nucleotides.

Sequence identity can be measured using sequence analysis software (for example, the Sequence Analysis Software Package of the Genetics Computer Group, University of Wisconsin Biotechnology Center, 1710 University Avenue, Madison, Wis. 53705), with the default parameters as specified therein.

The nucleic acid molecules of the invention can be inserted into a vector, as described below, which will facilitate expression of the insert. The nucleic acid molecules and the polypeptides they encode can be used directly as diagnostic or therapeutic agents, or can be used (directly in the case of the polypeptide or indirectly in the case of a nucleic acid molecule) to generate antibodies that, in turn, are clinically useful as a therapeutic or diagnostic agent. Accordingly, vectors containing the nucleic acid of the invention, cells transfected with these vectors, the polypeptides expressed, and antibodies generated, against either the entire polypeptide or an antigenic fragment thereof, are among the preferred embodiments.

The invention also features antibodies, e.g., monoclonal, polyclonal, and engineered antibodies, which specifically bind MAP44. By “specifically binds” is meant an antibody that recognizes and binds to a particular antigen, e.g., the MAP44 polypeptide of the invention, but which does not substantially recognize or bind to other mole-cules in a sample, e.g., a biological sample, which includes MAP44. References to constructs of antibody (or fragment thereof) coupled to a compound comprising a detectable marker includes constructs made by any technique, including chemical means or by recombinant techniques.

The invention also features antagonists and agonists of MAP44 that can inhibit or enhance one or more of the functions or activities of MAP44, respectively. Suitable antagonists can include small molecules (i.e., molecules with a molecular weight below about 500), large molecules (i.e., molecules with a molecular weight above about 500), antibodies that bind and “neutralize” MAP44 (as described below), polypeptides which compete with a native form of MAP44 for binding to a protein, e.g., MBL or ficolins, and nucleic acid molecules that interfere with transcription, of MAP44 (for example, antisense nucleic acid molecules and ribozymes). Agonists of MAP44 also include small and large molecules, and antibodies other than “neutralizing” antibodies.

The invention also features molecules, which can increase or decrease the expression of MAP44 (e.g., by influencing transcription or translation). Small molecules (i.e., molecules with a molecular weight below about 500), large molecules (i.e., molecules with a molecular weight above about 500), and nucleic acid molecules that can be used to inhibit the expression of MAP44 (for example, antisense and ribozyme molecules) or to enhance their expression (for example, expression constructs that place nucleic acid sequences encoding MAP44 under the control of a strong promoter system), and transgenic animals that express a MAP44 transgene.

The invention encompasses methods for treating disorders associated with aberrant expression or activity of MAP44. Thus, the invention includes methods for treating disorders associated with excessive expression or activity of MAP44. Such methods entail administering a compound, which decreases the expression or activity of MAP44. The invention also includes methods for treating disorders associated with insufficient expression of MAP44. These methods entail administering a compound, which increases the expression or activity of MAP44.

By “competitively inhibiting” serine protease activity is meant that, for example, the action of an altered MBL or ficolin or fragment thereof that can bind to a MAP44 peptide, reversibly or irreversibly without activating serine protease activity. Conversely, a fragment of MAP44, e.g., a polypeptide encompassing the N-terminal part of MAP44, may competitively inhibit the binding of the intact MAP44 and thus effectively inhibit the activity of MAP44.

The invention also features methods for detecting a MAP44 polypeptide. Such methods include: obtaining a biological sample; contacting the sample with an antibody that specifically binds MAP44 under conditions which permit specific binding; and detecting any antibody-MAP44 complexes formed.

In addition, the present invention encompasses methods and compositions for the diagnostic evaluation, typing, and prognosis of disorders associated with inappropriate expression or activity of MAP44. For example, the nucleic acid molecules of the invention can be used as diagnostic hybridization probes to detect, for example, inappropriate expression of MAP44 or mutations in the MAP44 gene. Such methods may be used to classify cells by the level of MAP44 expression.

Alternatively, the nucleic acid molecules can be used as primers for diagnostic PCR analysis for the identification of gene mutations, allelic variations and regulatory defects in the MAP44 gene. The present invention further provides for diagnostic kits for the practice of such methods.

The invention features methods of identifying compounds that modulate the expression or activity of MAP44 by assessing the expression or activity of MAP44 in the presence and absence of a selected compound. A difference in the level of expression or activity of MAP44 in the presence and absence of the selected compound indicates that the selected compound is capable of modulating expression or activity or MAP44. Expression can be assessed either at the level of gene expression (e.g., by measuring mRNA) or protein expression by techniques that are well known to skilled artisans. The activity of MAP44 can be assessed functionally, i.e., by assaying the activity of the compound.

The preferred methods and materials are described below in examples, which are meant to illustrate, not limit, the invention. Skilled artisans will recognize methods and materials that are similar or equivalent to those described herein, and that can be used in the practice or testing of the present invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the preferred methods and materials are described herein. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the detailed description, and from the description of the claims below.

Aspects of the invention concern a substantially pure mannan-binding lectin associated protein (MAP44) polypeptide. In some embodiments, this polypeptide is capable of associating with mannan-binding lectin (MBL) or one of the three ficolins, H-ficolin, L-ficolin and M-ficolin. Preferably, the polypeptide comprises an amino acid sequence identified as SEQ ID NO 1 or a functional equivalent of SEQ ID NO 1 and the polypeptide can be, optionally, conjugated to a label or toxin. The polypeptide can have a molecular mass of about 44 kDa under non-reducing conditions on an SDS-PAGE. The polypeptide can also be capable of MAP44 activity in an in vitro assay for MBL pathway or ficolin pathway function. The polypeptide can be capable of competitively inhibiting activity of MBL-associated serine proteases (MASPs) and fragments of the polypeptide of SEQ ID NO: 1 can be competitive inhibitors of the complexing of MBLIMASP or ficolin/MASP.

Aspects of the invention also include isolated nucleic acid molecules encoding the polypeptide above and the isolated nucleic acid molecules can comprise a nucleotide sequence encoding a polypeptide having sequence that is at least 50% identical to the sequence of SEQ ID NO:1. That is, the isolated nucleic acid sequence can encode a mannan-binding lectin associated protein (MAP44) having a polypeptide sequence at least 85% identical to SEQ ID NO:1. Stated differently, the isolated nucleic acid molecules can encode a mannan-binding lectin associated protein (MAp44) and said nucleic acid sequence can be at least 85% identical to SEQ ID NO:2. In some embodiments, these nucleic acids are incorporated into vectors, for example, expression vectors. In some embodiments, these nucleic acids comprise a regulatory element such as a promoter, that is operably linked thereto. Isolated cells comprising said isolated nucleic acids and vectors are also embodiments and said cells can be mammalian cells, a yeast cells, or a bacterial cells.

Aspects of the invention also include isolated antibodies, polyclonal and monoclonal, that are specific for said polypeptides. For instance, such an antibody can be produced by administering an MAP44 polypeptide, or part of the MAP44 polypeptide, or DNA encoding any such polypeptide, as described above, to an animal with the aim of producing antibody. Accordingly, aspects of the invention concern an antibody that selectively binds to MAP44 or a binding fragment thereof. Such an antibody can be a monoclonal antibody or a genetically engineered antibody or an antibody fragment and said molecules can be coupled to a compound comprising a detectable marker. Some embodiments also include pharmaceutical compositions comprising the polypeptides, nucleic acids, and antibodies, as described above. Such pharmaceuticals can be formulated for oral administration, injection, transdermal introduction, or inhalation.

Aspects of the invention also include methods for detecting mannan-binding lectin associated protein (MAP44) in a biological sample, said methods comprising: (a) obtaining a biological sample; (b) contacting said biological sample with a MAP44 polypeptide specific binding partner that specifically binds MAP44; and (c) detecting said complexes, if any, as an indication of the presence of mannan-binding lectin associated protein (MAP44) in said sample. By some approaches, the specific binding partner is an antibody to MAP44, as described above and in other embodiments, the specific binding partner is a mannan-binding lectin (MBL) or a ficolin. Additional embodiments concern methods for detecting MAP44, said methods comprising an assay for MAP44 activity, comprising the steps of a) applying a sample comprising MBLIMASP-2 complexes to a solid phase obtaining a bound complexes, b) applying MAP44 to the bound complexes, c) applying at least one complement factor to the complexes, d) detecting the amount of cleaved complement factors, e) correlating the amount of cleaved complement factors to the MAP44 activity. By some approaches, the solid phase is a mannan coating and by some approaches, the at least one complement factor is a complement factor cleavable by the MBLIMASP-2 complex, such as C3, C4, or C5, preferably C4. By some methodologies, the cleaved complement factor is detected by antibodies that are specific for the complement factor. In some assays, activation of the classical complement pathway is inhibited, for example; the activation is inhibited by conducting the assay at high ionic strength, such as a salt concentration is in the range of from 0.3 M to 10 M, such as from 0.5 M to 5 M, such as from 0.7 M to 2 M, such as from 0.9 M to 2 M, such as about 1.0 M. By some methods, the salt is selected from NaCI, KCI, MgCI2, or CaCI2. Preferably, the assays above are used to determine the amount of MAP44 in a biological sample or to determine the activity of MAP44 in a biological sample.

Additional approaches concern methods for detecting MAP44 nucleic acid expression, comprising detecting RNA having a sequence encoding a MAP44 polypeptide by mixing the sample with a nucleic acid probe that specifically hybridizes under high stringency conditions to the nucleic acid as described above (e.g., SEQ. ID. NO. 2).

Still more approaches concern methods treating patients that are deficient in MAP44 by administering to the patient a therapeutically effective amount of one or more of the polypeptides or nucleic acid or antibodies, or portions thereof described above. In some embodiments, said methods are practiced by administering one or more of the nucleic acids described above, such as an RNAi or antisense nucleic acid that complements SEQ ID NO. 2. In other embodiments, said methods are practiced by providing a compound that inhibits expression or activity of MAP44. In some embodiments said compound is an antibody or an antibody fragment or a peptidomimetic or aptamer corresponding to a molecule that binds MAP44. Preferably, said compound inhibits complexing of MBL and MAP44.

Additional embodiments concern assays that detect polymorphisms in the nucleic acid sequences encoding MAP44. Methods of detecting or identifying the presence of MAP44-encoding nucleic acid in a biological sample are also embodiments and said methods can be utilized by mixing the sample with at least one nucleic acid probe capable of forming a complex with MAP44-encoding nucleic acid under stringent conditions (preferably high stringency), and determining whether the probe is bound to sample nucleic acid. Accordingly, nucleic acid probes that are capable of forming a complex with MAP44-encoding nucleic acid under stringent conditions are also embodiments. That is, some embodiments include a nucleic acid sequence (e.g. an antisense sequence) capable of hybridizing to a nucleic acid sequence identical to SEQ ID NO 2 under high stringency conditions.

Additional embodiments concern assays that detect polymorphisms in the polypeptide sequence comprising MAP44 or its precursor. Methods for detecting or identifying a disorder associated with aberrant expression of MAP44 are also embodiments and said methods are utilized by obtaining a biological sample from a patient and measuring MAP44 expression in said biological sample, wherein increased or decreased MAP44 expression in said biological sample compared to a control indicates that said patient suffers from a disorder associated with aberrant expression of MAP44.

Methods for diagnosing, detecting, or identifying a disorder associated with aberrant activity of MAP44 can also be practiced by obtaining a biological sample from a patient and measuring MAP44 activity in said biological sample, wherein increased or decreased MAP44 activity in said biological sample compared to a control indicates that said patient suffers from a disorder associated with aberrant activity of MAP44.

As mentioned above, preferably the polypeptide described herein are used to make pharmaceutical compositions that can be administered parenterally, such as intramuscularly, intravenously, subcutaneously, or orally. Ideally, the pharmaceutical composition is formulated so that it is suitable for the treatment of a MAP44 deficiency, including, but not limited to an immunological disease. Accordingly, some embodiments include a composition for inhibiting complement activation encompassing a therapeutically effective amount of a MAP44 component and a pharmaceutically acceptable carrier. Methods of manufacturing said medicaments for use in inhibiting the effects of collectin complement activation in living subjects in need thereof are also embodiments and said methods can include the steps of combining a therapeutically effective amount of a MAP44 agent in a pharmaceutical carrier.

Methods of treating a subject suffering from a complement mediated vascular condition are also embodiments and said methods are practiced by administering an amount of a MAP44 agent effective to inhibit collectin-dependent complement activation. In some embodiments, the vascular condition is selected from the group consisting of a cardiovascular condition, a cerebrovascular condition, a peripheral (e.g., musculoskeletal) vascular condition, a renovascular condition, a mesenteric/enteric vascular condition, revascularization to transplants and/or replants, vasculitis, Henoch-Schonlein purpura nephritis, systemic lupus erythematosus-associated vasculitis, vasculitis associated with rheumatoid arthritis, immune complex vasculitis, Takayasu's disease, dilated cardiomyopathy, diabetic angiopathy, Kawasaki's disease (arteritis), venous gas embolus (VGE), and restenosis following stent placement, rotational atherectomy and percutaneous transluminal coronary angioplasty (PTCA).

Methods of treating a subject suffering from a collectin-dependent complement mediated condition associated with an ischemia-reperfusion injury are also embodiments and said methods can be practice by administering an amount of a MAP44 agent effective to inhibit collectin-dependent complement activation. In some embodiments, the ischemia-reperfusion injury is associated with aortic aneurysm repair, cardiopulmonary bypass, vascular reanastomosis in connection with organ transplants and/or extremity/digit replantation, stroke, myocardial infarction, and hemodynamic resuscitation following shock and/or surgical procedures.

Some embodiments also include methods of treating and/or preventing atherosclerosis in a subject in need thereof, comprising administering an amount of a MAP44 agent effective to inhibit collectin-dependent complement activation. Methods of treating a subject suffering from a collectin-dependent complement mediated condition associated with an inflammatory gastrointestinal disorder comprising administering an amount of a MAP44 agent effective to inhibit collectin-dependent complement activation are also encompassed by the present disclosure. In some of these methods, the inflammatory gastrointestinal disorder is selected from the group consisting of pancreatitis, Crohn's disease, ulcerative colitis, irritable bowel syndrome and diverticulitis. Methods of treating a subject suffering from a MASP-dependent complement mediated pulmonary condition are also embodiments and said methods are practiced by administering an amount of a MASP inhibitory agent effective to inhibit MASP-dependent complement activation. In some embodiments, the pulmonary condition is selected from the group consisting of acute respiratory distress syndrome, transfusionrelated acute lung injury, ischemiaireperfusion acute lung injury, chronic obstructive pulmonary disease, asthma, Wegener's granulomatosis, antiglomerular basement membrane disease (Goodpasture's disease), meconium aspiration syndrome, bronchiolitis obliterans syndrome, idiopathic pulmonary fibrosis, acute lung injury secondary to bum, non-cardiogenic pulmonary edema, transfusion-related respiratory depression and emphysema.

Methods of inhibiting MASP-dependent complement activation in a subject that has undergone, is undergoing, or will undergo an extracorporeal reperfusion procedure are also embodiments and said methods can be practiced by administering an amount of a MASP inhibitory agent effective to inhibit MASP-dependent complement activation. In some embodiments, the extracorporeal reperfusion procedure is selected from the group consisting of hemodialysis, plasmapheresis, leukopheresis, extracorporeal membrane oxygenator (ECMO), heparin-induced extracorporeal membrane oxygenation LDL precipitation (HELP) and cardiopulmonary bypass (CPB).

Methods of treating a subject suffering from a collectin-dependent complement mediated musculoskeletal condition are also embodiments and said methods can be practiced by administering an amount of a MAP44 agent effective to inhibit collectin-dependent complement activation. In some embodiments, the musculoskeletal condition is selected from the group consisting of osteoarthritis, rheumatoid arthritis, juvenile rheumatoid arthritis, gout, neuropathic arthropathy, psoriatic arthritis, spondyloarthropathy, crystalline arthropathy, muscular dystrophy and systemic lupus erythematosus (SLE).

Additional methods concern treating a subject suffering from a collectin-dependent complement mediated renal condition, wherein said subject is administered an amount of a MAP44 agent effective to inhibit collectin-dependent complement activation. In some embodiments, the renal condition is selected from the group consisting of mesangioproliferative glomerulonephritis, membranous glomerulonephritis, membranoproliferative glomerulonephritis (mesangiocapillary glomerulonephritis), acute postinfectious glomerulonephritis (post-streptococcal glomerulonephritis), cryoglobulinemic glomerulonephritis, lupus nephritis, Henoch-Schonlein purpura nephritis and IgA nephropathy.

More embodiments concern treating a subject suffering from a collectin-dependent complement mediated skin condition and said methods are practiced by administering an amount of a MAP44 agent effective to inhibit MASP-dependent complement activation. In some embodiments, the skin condition is selected from the group consisting of psoriasis, autoimmune bullous dermatoses, eosinophilic spongiosis, bullous pemphigoid, epidermolysis bullosa acquisita, herpes gestationis, thermal brn injury and chemical burn injury.

Still more embodiments concern methods of inhibiting collectin-dependent complement activation in a subject that has undergone, is undergoing, or will undergo an organ or tissue transplant procedure comprising administering an amount of a MAP44 agent effective to inhibit collectin-dependent complement activation. In some embodiments, the transplant procedure is selected from the group consisting of organ allotransplantation, organ xenotransplantation organ and a tissue graft. Methods of treating a subject suffering from a collectin-dependent complement mediated condition associated with a nervous system disorder or injury are also embodiments and said methods comprise administering an amount of a MASP inhibitory agent effective to inhibit MASP-dependent complement activation. In some embodiments, the nervous system disorder or injury is selected from the group consisting of multiple sclerosis, myasthenia gravis, Huntington's disease, amyotrophic lateral sclerosis, Guillain Bane syndrome, reperfusion following stroke, degenerative discs, cerebral trauma, Parkinson's disease, Alzheimer's disease, Miller-Fisher syndrome, cerebral trauma and/or hemorrhage, demyellination and meningitis.

Methods of treating a subject suffering from a collectin-dependent complement mediated condition associated with a blood disorder are also embodiments and said methods can comprise administering an amount of a MAP44 agent effective to inhibit MASP-dependent complement activation. In some embodiments, the blood disorder is selected from the group consisting of sepsis, severe sepsis, septic shock, acute respiratory distress syndrome resulting from sepsis, systemic inflammatory response syndrome, hemorrhagic shock, hemolytic anemia, autoimmune thrombotic thrombocytopenic purpura and hemolytic uremic syndrome.

Methods of treating a subject suffering from a collectin-dependent complement mediated condition associated with a urogenital condition are also embodiments and said methods can be practiced by administering an amount of a MAP44 agent effective to inhibit collectin-dependent complement activation. In some embodiments, the urogenital condition is selected from the group consisting of painful bladder disease, sensory bladder disease, chronic abacterial cystitis, interstitial cystitis, infertility, placental dysfunction and miscarriage and pre-eclampsia.

Methods of treating a subject suffering from a collectin-dependent complement mediated condition associated with nonobese diabetes (Type-I diabetes or Insulin-dependent diabetes mellitus) and/or complications associated with Type-1 or Type-2 (adult onset) diabetes are also encompassed by the present invention and said embodiments can be realized by administering an amount of a MAP44 agent effective to inhibit collectin-dependent complement activation. In some embodiments, the complication associated with Type 1 or Type 2 diabetes is selected from the group consisting of angiopathy, neuropathy and retinopathy.

Methods of inhibiting collectin-dependent complement activation in a subject that has undergone, is undergoing, or will undergo chemotherapeutic treatment and/or radiation therapy are also embodiments and said methods can be practiced by administering an amount of a MAP44 agent effective to inhibit collectin-dependent complement activation. Methods of treating a subject suffering from a malignancy are also embodiments and said methods can be practiced by administering an amount of a MAP44 agent effective to inhibit collectin-dependent complement activation.

Methods of treating a subject suffering from an endocrine disorder are also embodiments and said methods can be practiced by administering an amount of a MAP44 agent effective to inhibit collectin-dependent complement activation. In some embodiments, the endocrine disorder is selected from the group consisting of Hashimoto's thyroiditis, stress, anxiety, hormonal disorders involving regulated release of prolactin, growth or other insulin-like growth factor and adrenocorticotropin from the pituitary. Methods of inhibiting the activation of protease activated receptor 4 (PAR4) are also embodiments and said methods can be practiced by administering an amount of a MAP44 agent effective to inhibit MASP1-dependent PAR4 cleaving activity. Methods of treating a subject suffering from obesity are also embodiments and said methods can be practiced by administering an amount of a MAP44 agent effective to inhibit MASP1-dependent cleaving of complement factor D. Methods of treating a subject suffering from a complement mediated ophthalmologic condition are also embodiments and said methods can be practiced by administering an amount of a MAP44 agent effective to inhibit MASP-dependent complement activation. In some embodiments, the ophthalmologic condition is age-related macular degeneration.

Preferred embodiments include isolated polypeptides that comprise:

(a) the amino acid sequence of SEQ ID NO 1;

(b) an amino acid sequence with at least about 85%, 90%, 95%, 98%, or 99% sequence identity to SEQ. ID. NO. 1, wherein said polypeptide is capable of binding to mannan-binding lectin (MBL), H-ficolin, L-ficolin, or M-ficolin; or

(c) a fragment of SEQ. ID. NO. 1 that is at least 50, 60, 70, 80, 90, or 100 amino acids in length, wherein said fragment is capable of competitively inhibiting MBL-associated serine proteases (MASPs) or the formation of the MBLIMASP or ficolin/MASP complex.

Any one or more of the isolated polypeptides above can further comprise a label or toxin joined thereto.

Additional preferred embodiments include isolated nucleic acids that comprise:

(a) a nucleotide sequence that encodes SEQ ID NO 1;

(b) the nucleotide sequence of SEQ ID NO 2;

(c) a fragment of a nucleotide sequence of SEQ ID NO 2 that is at least about 50, 60, 70, 80, 90, 100, 125, 150, or 200 nucleotides in length; or

(d) a nucleotide sequence with at least about 85%, 90%, 95%, 98%, or 99% sequence identity to SEQ. ID. NO. 2, wherein the polypeptide encoded by said nucleic acid is capable of binding to mannan-binding lectin (MBL), H-ficolin, L-ficolin, or M-ficolin.

Any one or more of the isolated nucleic acids above can further comprise a vector sequence, such as an expression vector sequence or a regulatory element linked operably thereto.

Additional preferred embodiments include methods for identifying the presence of MAP44 in a biological sample and said methods can be practiced by detecting the amount of a polypeptide comprising SEQ ID NO 1 or the amount of an RNA encoding SEQ ID NO 1 in a biological sample. In some embodiments, the amount of the polypeptide comprising SEQ ID NO 1 or the amount of the RNA encoding SEQ ID NO 1 detected with a reference amount of MAP44 or an RNA encoding MAP44 or the amount of the polypeptide comprising SEQ ID NO 1 or the amount of the RNA encoding SEQ ID NO 1 detected in a second biological sample.

### BRIEF DESCRIPTION OF THE DRAWINGS

FIG. S1. Agarose gel analysis of MAp44 mRNA specific PCRs on various brain-derived cell-lines and human brain cDNA. A, PCR performed using the MAp44-specific primer pair, designed to specifically amplify a 497 bp segment from only mature MAp44 mRNA. B, PCR performed using the MASP1 common primer pair, designed to amplify a 435 bp segment of the common part of mature MASP-1, MASP-3, and MAp44 mRNAs. The cDNA used as template in each reaction is indicated at the top of each lane, and relative positions of molecular size markers are indicated on the sides. The black lines indicate excision of irrelevant lanes.

FIG. S2. Staining of Western blots with anti-MAp44 antibodies. A, a blot of reduced MBL/MASP complex from human plasma developed with polyclonal anti-MAp44 antiserum from rabbit R74B, affinity-purified antibody and, as a control, the pre-immunization serum from the same animal. Rabbit anti-MASP-3 SP domain antiserum (R32) and rabbit serum R64 recognizing MASP-1 SP domain was also tested. B, a blot of reduced rMAp44-containing culture supernatant was developed with R74B immune serum, affinity-purified antibody and pre-immune serum, and with mAb anti-MASP-1/-3 A-chain antibody (1E2). C, blots of purified MBL/MASP complex and rMAp44 supernatant run under non-reducing conditions and developed with 1E2. Molecular weight markers are indicated on the sides.

FIG. S3. MBL and ficolins were caught from human serum in microtiter wells coated with anti-MBL, anti-L-ficolin or anti-H-ficolin antibody. Non-specific monoclonal IgG1 served as control. Bound MBL or ficolins, together with associated proteins, were eluted with SDS-sample buffer, applied to reducing SDS-PAGE and analyzed by Western blotting using monoclonal mouse anti-MASP-2/MAp19 antibody (1.3B7). Sample ID for each lane is denoted at the top. Purified MBL/MASP complexes (positive control) were also tested. The black lines indicate excision of irrelevant lanes. The Mw in kDa of each band of the markers is given on the sides.

FIG. 1. Genomic organization, splice pattern and protein structures. A, exon-intron structure of the MASP1 gene encoding MASP-1, MASP-3 and MAP44. Protein encoding regions are white boxes, 5′ and 3′ UTRs are gray. Intron sizes are not to scale. The asterisks indicate potential N-linked glycosylation sites. Exon 1-8, 10 and 11 encode the identical A-chain of MASP-1 and -3. Exon 12 and exons 13-18 encode the serine protease domains of MASP-3 and -1, respectively. The pre-mRNA is spliced differentially to yield the mRNAs encoding the 380 amino acid residue long MAP44, encompassing the signal peptide, the domains CUB1-EGF-CUB2-CCP1, and 17 extra residues, and the mRNAs for MASP-1 and MASP-3 encompassing the signal peptide and 6 domains (CUB1-EGF-CUB2-CCP1-CCP2, serine protease domain) as well as the activation peptide region (AP). The 17 unique amino acids of MAP44 are encoded by exon 9 located between two of the shared exons of MASP-1 and -3. B, intron-exon boundaries governing the alternative splice events of MASP-1/-3 vs. MAP44 mRNA. The nucleotides surrounding the splice donor and acceptor sites for each of the three introns are indicated. Sequences conforming to the gt/ag rule are shown in bold typeface. Exons are shown in uppercase and introns in lowercase letters. The underlined sequence indicates the predicted optimal branch site (consensus: CTRAYY (SEQ ID NO 3).

FIG. 2. Expression of mRNA encoding MAP44, MASP-3 and MASP-1 in human tissues. mRNA levels were determined by qRT-PCR. The source of the RNA is given below the bars and the relative mRNA level on the y-axis. The values obtained from liver RNA were set to 1,000 units. A, B, and C, show MAP44, MASP-3 and MASP-1 mRNA levels, respectively. The experiment was performed three times with similar results, each time using 2 and 20 ng of template cDNA.

FIG. 3. Association of MAP44 with MBL and ficolins in serum. A, MBL and ficolins were captured from human serum in microtiter wells coated with anti-MBL, anti-L-ficolin or anti-H-ficolin antibody. Non-specific monoclonal IgG₁ served as control. Bound MBL or ficolins, together with associated proteins, were eluted with SDS-sample buffer, applied to non-reducing SDS-PAGE and analyzed by Western blotting using rabbit anti-MAP44 antibody. Sample ID for each lane is denoted at the top. Purified MBL/MASP complexes (positive control) were also tested. The black lines indicate excision of irrelevant lanes. The M_{w} in kDa of each band of the marker is given on the right side. The experiment was repeated twice with similar results. B, MBL or ficolins were captured in microtiter wells and specific antibodies were used to detect MAP44. The capture antibodies are given below the x-axis. The signal was detected by time-resolved fluorometry and is given on the y-axis as counts per second. The error bars indicate the standard deviation of duplicate measurements. C, similar to B, but in this case development was with anti-MASP-3 antibody.

FIG. 4. Surface plasmon resonance measurements of the interactions between MAP44 and MBL, and between MASP-3 and MBL. A, silver staining of an SDS-PAGE gel of the purified rMAP44 and rMASP-3 used. B, sensorgrams for the interaction of rMAP44 analyte at concentrations from 1-30 nM with a fixed amount of rMBL ligand coated on the chip. C, sensorgrams for the interaction of rMASP-3 analyte at concentrations from 1-30 nM on the same surface as in panel B.

FIG. 5. GPC analysis of the distribution of MAP44 in human serum. Serum (100 μl) was passed through a Superose 6 column and fractions were analyzed for MAP44 content by TRIFMA. The serum was fractionated in an isotonic Ca²⁺-containing buffer (▾) or in a high salt and EDTA-containing buffer (conditions dissociating MBL/MASP complexes) (▪). Arrows indicate the elution volumes of IgM (970 kDa), IgG (150 kDa) and HSA (67 kDa). The elution positions of MBL and ficolins in Ca²⁺-containing buffer are also indicated. The experiment was repeated twice with similar results.

FIG. 6. MAP44-mediated inhibition of MASP binding and of complement activation. A, competition between MAP44 and MASP-3 for binding to MBL. Constant concentrations of rMAP44 and rMBL were incubated with increasing concentrations of rMASP-3. After incubation, the MBL-containing complexes were captured in microtiter wells coated with mannan, and bound MAP44 was detected with anti-MAP44 antibody and bound MASP-3 with anti-MASP-3 antibody. The amounts of bound rMAP44 () and MASP-3 (▾) are plotted on the left and right-hand side y-axis, respectively. B, inhibition of MBL/MASP-2 mediated C4 deposition. A mixture of rMBL and rMASP-2 was incubated with rMAP44 () or rMAp19 (▴) at increasing concentrations and subsequently incubated in mannan-coated wells. The wells were next incubated with purified human C4, followed by detection of deposited C4 fragments by anti-C4 antibody. C, inhibition of MASP-2 binding to MBL as a function of pre-incubation with increasing concentrations of competitor. A constant concentration of rMBL and rMASP-2 was incubated with increasing amounts of rMAp44 () or rMAP19 (▴). Following incubation in mannan-coated microliter wells the wells were developed with anti-MASP-2 antibody.

FIG. 7. Phylogram based on MAP44 sequence similarity between various vertebrate species and a urochordate. A complete alignment of the full-length peptide sequences of MAP44 from human, chimpanzee, rhesus macaque, long-tailed macaque, cow, dog, mouse, rat, chicken, lizard, African clawed frog, zebrafish, carp and sea squirt was created using ClustalX v. 2.0.10 with default settings and iteration at each alignment step. Based on this alignment a consensus bootstrapped N-J tree was produced, excluding positions with gaps and omitting correction for multiple substitutions. The tree was rooted in FigTree v. 1.2.1 using C. intestinalis as out-group. Bootstrap values out of 1000 are given on nodes and percentage similarity values to human MAP44 are given in parenthesis after node labels.

TABLE SI. MAp44 sequence similarity across various species (% identity).

### DETAILED DESCRIPTION OF THE INVENTION

### MAP44 Nucleic Acid Molecules

The MAP44 nucleic acid molecules of the invention can be cDNA, genomic DNA, synthetic DNA, or RNA, and can be double-stranded or single-stranded (i.e., either a sense or an antisense strand). Fragments of these molecules are also considered within the scope of the invention, and can be produced, for example, by the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide (for example, the polypeptide of SEQ ID NO:1). In addition, these nucleic acid molecules are not limited to sequences that only encode polypeptides, and thus, can include some or all of the non-coding sequences that lie upstream or downstream from a coding se-quence.

In a preferred embodiment the invention relates to an isolated nucleic acid molecule encoding the polypeptide defined herein, the molecule comprising a nucleotide sequence encoding a polypeptide having sequence that is at least 50% identical to the sequence of SEQ ID NO:1. The polypeptide is preferably a mannan-binding lectin associated protein (MAP44) having a polypeptide sequence at least 85% identical to SEQ ID NO:1.

Thus, the isolated nucleic acid sequence preferably encodes a mannan-binding lectin associated protein (MAP44), said nucleic acid sequence being at least 85% identical to SEQ ID NO:2.

The nucleic acid molecules of the invention can be synthesized (for example, by phosphoramidite-based synthesis) or obtained from a biological cell, such as the cell of a mammal. Thus, the nucleic acids can be those of, e.g., a human, mouse, rat, guinea pig, cow, sheep, horse, pig, rabbit, monkey, dog, or cat. Combinations or modifications of the nucleotides within these types of nucleic acids are also encompassed.

In addition, the isolated nucleic acid molecules of the invention encompass fragments that are not found as such in the natural state. Thus, the invention encompasses recombinant molecules, such as those in which a nucleic acid molecule (for example, an isolated nucleic acid molecule encoding MAP44) is incorporated into a vector (for example, a plasmid or viral vector) or into the genome of a heterologous cell (or the genome of a homologous cell, at a position other than the natural chromosomal location). Recombinant nucleic acid molecules and uses therefore are discussed further below.

In the event the nucleic acid molecules of the invention encode or act as antisense molecules, they can be used for example, to regulate translation of MAP44. Techniques associated with detection or regulation of nucleic acid expression are well known to skilled artisans and can be used to diagnose and/or treat disorders associated with MAP44 activity. These nucleic acid molecules are discussed further below in the context of their clinical utility.

The invention also encompasses nucleic acid molecules that hybridize under stringent conditions to a nucleic acid molecule encoding a MAP44 polypeptide. The cDNA sequence described herein (SEQ ID NO:2) can be used to identify these nucleic acids, which include, for example, nucleic acids that encode homologous polypeptides in other species, and splice variants of the MAP44 gene in humans or other mammals. Accordingly, the invention features methods of detecting and isolating these nucleic acid molecules.

Using these methods, a sample (for example, a nucleic acid library, such as a cDNA or genomic library) is contacted (or “screened”) with a MAP44-specific probe (for example, a fragment of SEQ ID NO:2 that is at least 12 nucleotides long). The probe will selectively hybridize to nucleic acids encoding related polypeptides (or to complementary sequences thereof). Because the polypeptide encoded by MAP44 is related to other proteins, the term “selectively hybridize” is used to refer to an event in which a probe binds to nucleic acids encoding MAP44 (or to complementary sequences thereof) to a detectably greater extent than to nucleic acids encoding other proteins (or to complementary sequences thereof). The probe, which can contain at least 12 (for example, 15, 25, 50, 100, or 200 nucleotides) can be produced using any of several standard methods (see, for example, Ausubel et al., “Current Protocols in Molecular Biology”, John Wiley & Sons, Inc., NY, 2007). For example, the probe can be generated using PCR amplification methods in which oligonucleotide primers are used to amplify a MAP44-specific nucleic acid sequence (for example, a nucleic acid encoding the N-terminus of mature MAP44) that can be used as a probe to screen a nucleic acid and thereby detect nucleic acid molecules (within the library) that hybridize to the probe.

One single-stranded nucleic acid is said to hybridize to another if a duplex forms between them. This occurs when one nucleic acid contains a sequence that is the reverse and complement of the other (this same arrangement gives rise to the natural interaction between the sense and antisense strands of DNA in the genome and underlies the configuration of the “double helix”). Complete complementarity between the hybridizing regions is not required in order for a duplex to form; it is only necessary that the number of paired bases is sufficient to maintain the duplex under the hybridization conditions used.

In one aspect, the invention relates to a nucleic acid probe capable of forming a complex with MAP44-encoding nucleic acid under stringent conditions, such as a sequence capable of hybridizing to a nucleic acid sequence identical to SEQ ID NO 2.

The hybridizable probe may be an anti-sense nucleic acid with respect to a nucleic acid sequence encoding MAP44.

Typically, hybridization conditions are of low to moderate stringency. These conditions favour specific interactions between completely complementary sequences, but allow some non-specific interaction between less than perfectly matched sequences to occur as well. After hybridization, the nucleic acids can be “washed” under moderate or high conditions of stringency to dissociate duplexes that are bound together by some non-specific interaction (the nucleic acids that form these duplexes are thus not completely complementary).

As is known in the art, the optimal conditions for washing are determined empirically, often by gradually increasing the stringency. The parameters that can be changed to affect stringency include, primarily, temperature and salt concentration. In general, the lower the salt concentration and the higher the temperature, the higher the stringency. Washing can be initiated at a low temperature (for example, room temperature) using a solution containing a salt concentration that is equivalent to or lower than that of the hybridization solution. Subsequent washing can be carried out using progressively warmer solutions having the same salt concentration. As alternatives, the salt concentration can be lowered and the temperature maintained in the washing step, or the salt concentration can be lowered and the temperature increased. Additional parameters can also be altered. For example, use of a destabilizing agent, such as formamide, alters the stringency conditions.

In reactions where nucleic acids are hybridized, the conditions used to achieve a given level of stringency will vary. There is not one set of conditions, for example, that will allow duplexes to form between all nucleic acids that are 85% identical to one another; hybridization also depends on unique features of each nucleic acid. The length of the sequence, the composition of the sequence (for example, the content of purine-like nucleotides versus the content of pyrimidine-like nucleotides) and the type of nucleic acid (for example, DNA or RNA) affect hybridization. An additional consideration is whether one of the nucleic acids is immobilized (for example, on a filter).

An example of a progression from lower to higher stringency conditions is the following, where the salt content is given as the relative abundance of SSC (a salt solution containing sodium chloride and sodium citrate; 2×SSC is 10-fold more concentrated than 0.2×SSC). Nucleic acids are hybridized at 42° C. in 2×SSC/0.1% SDS (sodium dodecylsulfate; a detergent) and then washed in 0.2×SSC/0.1% SDS at room temperature (for conditions of low stringency); 0.2×SSC/0.1% SDS at 42° C. (for conditions of moderate stringency); and 0.1×SSC at 68° C. (for conditions of high stringency). Washing can be carried out using only one of the conditions given, or each of the conditions can be used (for example, washing for 10-15 minutes each in the order listed above). Any or all of the washes can be repeated. As mentioned above, optimal conditions will vary and can be determined empirically.

A second set of conditions that are considered “stringent conditions” are those in which hybridization is carried out at 50° C. in Church buffer (7% SDS, 0.5% NaHPO4, 1 M EDTA, 1% bovine serum albumin) and washing is carried out at 50° C. in 2×SSC.

Once detected, the nucleic acid molecules can be isolated by any of a number of standard techniques (see, for example, Sambrook et al., “Molecular Cloning, A Laboratory Manual,” Third edition. Cold Spring Harbor Laboratory Press, Cold Spring Harbor, N.Y., 2001).

The invention also encompasses: (a) expression vectors that contain any of the foregoing MAP44-related coding sequences and/or their complements (that is, “antisense” sequence); (b) expression vectors that contain any of the foregoing MAP44-related coding sequences operatively associated with a regulatory element (examples of which are given below) that directs the expression of the coding sequences; (c) expression vectors containing, in addition to sequences encoding a MAP44 poly-peptide, nucleic acid sequences that are unrelated to nucleic acid sequences encoding MAP44, such as molecules encoding a reporter or marker; and (d) genetically engineered host cells that contain any of the foregoing expression vectors and thereby express the nucleic acid molecules of the invention in the host cell.

Recombinant nucleic acid molecule can contain a sequence encoding a soluble MAP44, mature MAP44, MAP44 having a signal sequence, or functional domains of MAP44 such as a CUB domain, an EGF domain, or a MBL-binding or ficolin-binding domain. The full length MAP44 polypeptide, a domain of MAP44, or a fragment thereof may be fused to additional polypeptides, as described below. Similarly, the nucleic acid molecules of the invention can encode the mature form of MAP44 or a form that encodes a polypeptide which facilitates secretion. In the latter instance, the polypeptide is typically referred to as a proprotein, which can be converted into an active form by removal of the signal sequence, for example, within the host cell. Proproteins can be converted into the active form of the protein by removal of the inactivating sequence.

The regulatory elements referred to above include, but are not limited to, inducible and non-inducible promoters, enhancers, operators and other elements, which are known to those skilled in the art, and which drive or otherwise regulate gene expression. Such regulatory elements include but are not limited to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast-mating factors.

Similarly, the nucleic acid can form part of a hybrid gene encoding additional polypeptide sequences, for example, sequences that function as a marker or reporter. Examples of marker or reporter genes include—lactamase, chloramphenicol acetyl-transferase (CAT), adenosine deaminase (ADA), aminoglycoside phosphotransferase (neo^{r}, G418^{r}), dihydrofolate reductase (DHFR), hygromycin-B-phosphotrans-ferase (HPH), thymidine kinase (TK), lacZ (encoding-galactosidase), green fluorescent protein (GFP), and xanthine guanine phosphoribosyltransferase (XGPRT). As with many of the standard procedures associated with the practice of the invention, skilled artisans will be aware of additional useful reagents, for example, of additional sequences that can serve the function of a marker or reporter. Generally, the hybrid polypeptide will include a first portion and a second portion; the first portion being a MAP44 polypeptide and the second portion being, for example, the reporter described above or an immunoglobulin constant region.

The expression systems that may be used for purposes of the invention include, but are not limited to, microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecules of the invention (preferably containing nucleic acid sequences of MAP44 (SEQ ID NO:2)); insect cell systems infected with recombinant virus expression vectors (for example, baculovirus) containing the nucleic acid molecules of the invention; plant cell systems infected with recombinant virus expression vectors (for example, cauliflower mosaic virus (CaMV) and tobacco mosaic virus (TMV)) or transformed with recombinant plasmid expression vectors (for example, Ti plasmid) containing MAP44 nucleotide sequences; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metalothionein promoter) or from mammalian viruses (for example, the adenovirus late promoter and the vaccinia virus 7.5K promoter).

In bacterial systems, a number of expression vectors may be advantageously selected depending upon the use intended for the gene product being expressed. For example, when a large quantity of such a protein is to be produced, for the generation of pharmaceutical compositions containing MAP44 polypeptides or for raising antibodies to those polypeptides, vectors that are capable of directing the expression of high levels of fusion protein products that are readily purified may be desirable. Such vectors include, but are not limited to, the E. coli expression vector pUR278 (Ruther et al., EMBO J. 2:1791, 1983), in which the coding sequence of the insert may be ligated individually into the vector in frame with the lacZ coding region so that a fusion protein is produced; pIN vectors (Inouye and Inouye, Nucleic Acids Res. 13:3101-3109, 1985; Van Heeke and Schuster, J. Biol. Chem. 264:5503-5509, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) can be used as a vector to express foreign genes. The virus grows in Spodoptera frugiperda cells. The coding sequence of the insert may be cloned individually into non-essential regions (for example the polyhedrin gene) of the virus and placed under control of an AcNPV promoter (for example the polyhedrin promoter). Successful insertion of the coding sequence will result in inactivation of the polyhedrin gene and production of non-occluded recombinant virus (i.e., virus lacking the proteina-ceous coat coded for by the polyhedrin gene). These recombinant viruses are then used to infect Spodoptera frugiperda cells in which the inserted gene is expressed. (for example, see Smith et al., J. Virol. 46:584, 1983; Smith, U.S. Pat. No. 4,215,051).

In mammalian host cells, a number of viral-based expression systems may be utilized. In cases where an adenovirus is used as an expression vector, the nucleic acid molecule of the invention may be ligated to an adenovirus transcription/translation control complex, for example, the late promoter and tripartite leader sequence. This chimeric gene may then be inserted in the adenovirus genome by in vitro or in vivo recombination. Insertion in a non-essential region of the viral genome (for example, region E1 or E3) will result in a recombinant virus that is viable and capable of expressing a MAP44 gene product in infected hosts (for example, see Logan and Shenk, Proc. Natl. Acad. Sci. USA 81:3655-3659, 1984). Specific initiation signals may also be required for efficient translation of inserted nucleic acid molecules. These signals include the ATG initiation codon and adjacent sequences. In cases where an entire gene or cDNA, including its own initiation codon and adjacent sequences, is inserted into the appropriate expression vector, no additional translational control signals may be needed. However, in cases where only a portion of the coding sequence is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544, 1987).

In addition, a host cell strain may be chosen which modulates the expression of the inserted sequences, or modifies and processes the gene product in the specific fashion desired. Such modifications (for example, glycosylation) and processing (for example, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells, which possess the cellular machinery for proper processing of the primary transcript, glycosylation, and phosphorylation of the gene product may be used. The mammalian cell types listed above are among those that could serve as suitable host cells.

For long-term, high-yield production of recombinant proteins, stable expression is preferred. For example, cell lines which stably express the MAP44 sequences described above may be engineered. Rather than using expression vectors which contain viral origins of replication, host cells can be transformed with DNA controlled by appropriate expression control elements (for example, promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express MAP44.

Such engineered cell lines may be particularly useful in screening and evaluation of compounds that affect the endogenous activity of the gene product and for production of MAP44 for therapeutic uses. These methods may also be used to modify cells that are introduced into a host organism either for experimental or therapeutic purposes. The introduced cells may be transient or permanent within the host organism.

A number of selection systems can be used. For example, the herpes simplex virus thymidine kinase (Wigler, et al., Cell 11:223, 1977), hypoxanthine guanine phosphoribosyltransferase (Szybalska and Szybalski, Proc. Natl. Acad. Sci. USA 48:2026, 1962), and adenine phosphoribosyltransferase (Lowy, et al., Cell 22:817, 1980) genes can be employed in tk−, hgprt− or aprt− cells, respectively. Also, anti-metabolite resistance can be used as the basis of selection for the following genes: dhfr, which confers resistance to methotrexate (Wigler et al., Proc. Natl. Acad. Sci. USA 77:3567, 1980; O'Hare et al., Proc. Natl. Acad. Sci. USA 78:1527, 1981); gpt, which confers resistance to mycophenolic acid (Mulligan and Berg, Proc. Natl. Acad. Sci. USA 78:2072, 1981); neo, which confers resistance to the aminoglycoside G-418 (Colberre-Garapin et al., J. Mol. Biol. 150:1, 1981); and hygro, which confers resistance to hygromycin (Santerre et al., Gene 30:147, 1984).

Alternatively, any fusion protein may be readily purified by utilizing an antibody specific for the fusion protein being expressed. For example, a system described by Janknecht et al. allows for the ready purification of non-denatured fusion proteins expressed in human cell lines (Proc. Natl. Acad. Sci. USA 88: 8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni²⁺. nitriloacetic acid-agarose columns and histidine-tagged proteins are selectively eluted with imidazole-containing buffers.

### MAP44 Polypeptides

The MAP44 polypeptides described herein are those encoded by any of the nucleic acid molecules described above and include MAP44 fragments, mutants, truncated forms, and fusion proteins. These polypeptides can be prepared for a variety of uses, including but not limited to the generation of antibodies, as reagents in diagnostic assays, for the identification of other cellular gene products or compounds that can modulate the collectin response, and as pharmaceutical reagents useful for the treatment of inflammation and certain disorders (described below) that are associated with activity of the lectin pathway. Preferred polypeptides are substantially pure MAP44 polypeptides, including those that correspond to the polypeptide with an intact signal sequence, the mature form of the polypeptide of the human MAP44 polypeptide as well as polypeptides representing a part of the MAP44 polypeptide. Especially preferred are polypeptides that are soluble under normal physiological conditions.

In particular the invention relates to polypeptides comprising an amino acid sequence identified as SEQ ID NO 1 or a functional equivalent of SEQ ID NO 1, and/or an amino acid sequence identified as SEQ ID NO 1 or a functional equivalent of SEQ ID NO 1.

In one embodiment the polypeptide may be defined as a polypeptide having a molecular mass of about 44 kDa under non-reducing conditions on an SDS-PAGE, such as a polypeptide containing the sequence identified as SEQ ID NO 1.

The invention also encompasses polypeptides that are functionally equivalent to MAP44. These polypeptides are equivalent to MAP44 in that they are capable of carrying out one or more of the functions of MAP44 in a biological system. Preferred MAP44 polypeptides have 20%, 40%, 50%, 75%, 80%, or even 90% of the activity of the full-length, mature human form of MAP44. Such comparisons are generally based on an assay of biological activity in which equal concentrations of the polypeptides are used and compared. The comparison can also be based on the amount of the polypeptide required to reach 50% of the maximal activity obtainable.

Functionally equivalent proteins can be those, for example, that contain additional or substituted amino acid residues. Substitutions may be made on the basis of similarity in polarity, charge, solubility, hydrophobicity, hydrophilicity, and/or the amphipathic nature of the residues involved. Amino acids that are typically considered to provide a conservative substitution for one another are specified in the summary of the invention. D-amino acids may be introduced in order to modify the half-life of the polypeptide.

Polypeptides that are functionally equivalent to MAP44 (e.g. SEQ ID NO:1) can be made using random mutagenesis techniques well known to those skilled in the art (and the resulting mutant MAP44 proteins can be tested for activity). It is more likely, however, that such polypeptides will be generated by site-directed mutagenesis (again using techniques well known to those skilled in the art). These polypeptides may have an increased function, i.e., a greater ability to inactivate the lectin pathway. Such polypeptides can be used to inhibit the activity of lectin pathway immune function.

To design functionally equivalent polypeptides, it is useful to distinguish between conserved positions and variable positions. This can be done by aligning the sequence of MAP44 cDNAs that were obtained from various organisms. Skilled arti-sans will recognize that conserved amino acid residues are more likely to be necessary for preservation of function.

Mutations within the MAP44 coding sequence can be made to generate MAP44 peptides that are better suited for expression in a selected host cell. Introduction of a glycosylation sequence can also be used to generate a MAP44 polypeptide with altered biological characteristics.

The invention also features methods for assay of polymorphisms within the polypeptide sequence comprising MAP44 or its precursor. This may be accomplished by a number of techniques. For example, the purified polypeptide is subjected to tryptic digestion and the resulting fragments analyzed by either one- or two-dimensional electrophoresis. The results from analysis of a sample polypeptide are compared to the results using a known sequence. Also the analysis may encompass separation of a biological sample (e.g., serum or other body fluids) by either one- or two-dimensional electrophoresis followed by transfer of the separated proteins onto a membrane (western blot). The membrane is then reacted with antibodies against MAP44, followed by a secondary labelled antibody. The staining pattern is corn-pared with that obtained using a sample with a known sequence or modification.

The polypeptides of the invention can be expressed fused to another polypeptide, for example, a marker polypeptide or fusion partner. For example, the polypeptide can be fused to a hexahistidine tag to facilitate purification of bacterially expressed protein or a hemagglutinin tag to facilitate purification of protein expressed in eukaryotic cells. The MAP44 polypeptide of the invention, or a portion thereof, can also be altered so that it has a longer circulating half-life by fusion to an immunoglobulin Fc domain (Capon et al., Nature 337:525-531, 1989). Similarly, a dimeric form of the MAP44 polypeptide can be produced, which has increased stability in vivo.

In order to use the polypeptide for diagnostic purposes the polypeptide may be conjugated to a label or toxin.

The polypeptides of the invention can be chemically synthesized (for example, see Creighton, “Proteins: Structures and Molecular Principles,” W.H. Freeman & Co., NY, 1983), or, perhaps more advantageously, produced by recombinant DNA technology as described herein. For additional guidance, skilled artisans may consult Ausubel et al. (supra), Sambrook et al. (“Molecular Cloning, A Laboratory Manual,” Cold Spring Harbor Press, Cold Spring Harbor, N.Y., 2001), and, particularly for examples of chemical synthesis Gait, M. J. Ed. (“Oligonucleotide Synthesis,” IRL Press, Oxford, 1984).

The invention also features polypeptides that interact with MAP44 (and the genes that encode them) and thereby alter the function of MAP44 interacting polypeptides can be identified using methods known to those skilled in the art. One suitable method is the “two-hybrid system,” which detects protein interactions in vivo (Chien et al., Proc. Natl. Acad. Sci. USA, 88:9578, 1991). A kit for practicing this method is available from Clontech (Palo Alto, Calif.).

### Anti-MAP44 Antibodies

Human MAP44 polypeptides (or immunogenic fragments or analogs) can be used to raise antibodies useful in the invention; such polypeptides can be purified from se-rum or tissues, produced by recombinant techniques or synthesized (see, for example, “Solid Phase Peptide Synthesis,” supra; Ausubel et al., supra). In general, the peptides can be coupled to a carrier protein, such as KLH, as described in Ausubel et al., supra, mixed with an adjuvant, and injected into a host mammal. Also the carrier could be PPD. Antibodies can be purified by peptide antigen affinity chromatography.

In particular, various host animals can be immunized by injection with a MAP44 protein or polypeptide. Host animals include rabbits, mice, guinea pigs, rats, and chickens. Various adjuvants that can be used to increase the immunological response depend on the host species and include Freund's adjuvant (complete and incomplete), mineral gels such as aluminum hydroxide, surface-active substances such as lysolecithin, pluronic polyols, polyanions, peptides, oil emulsions, keyhole limpet hemocyanin, and dinitrophenol. Potentially useful human adjuvants include BCG (bacille Calmette-Guerin) and Corynebacterium parvum. Immunizations may also be carried out by the injection of DNA encoding MAP44 or parts thereof. Polyclonal antibodies are heterogeneous populations of antibody molecules that are contained in the sera of the immunized animals.

The invention preferably relates to an antibody produced by administering an MAP44 polypeptide, or part of the MAP44 polypeptide, or DNA encoding any such polypeptide, according to claim 1 to an animal with the aim of producing antibody. It is preferred that said antibody selectively binds to MAP44.

Antibodies within the invention therefore include polyclonal antibodies and, in addition, monoclonal antibodies, humanized or chimeric or fully human antibodies, single chain antibodies, Fab fragments, F(ab′)₂ fragments, and molecules produced using a Fab expression library, and antibodies or fragments produced by phage display techniques.

Monoclonal antibodies, which are homogeneous populations of antibodies to a particular antigen, can be prepared using the MAP44 proteins described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, NY, 1981; Ausubel et al., supra).

In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp. 77-96, 1983). Such antibodies can be of any immunoglobulin class including IgG, IgM, IgE, IgA, IgD and any subclass thereof. (In the case of chickens, the immunoglobulin class can also be IgY.) The hybridoma producing the mAb of this invention may be cultivated in vitro or in vivo. The ability to produce high titers of mAbs in vivo makes this the presently preferred method of production, but in some cases, in vitro production will be preferred to avoid introducing cancer cells into live animals, for example, in cases where the presence of normal immunoglobulins coming from the acitis fluids are unwanted, or in cases involving ethical considerations.

Once produced, polyclonal, monoclonal, or phage-derived antibodies are tested for specific MAP44 recognition by Western blot or immuno-precipitation analysis by standard methods, e.g., as described in Ausubel et al., supra. Antibodies that specifically recognize and bind to MAP44 are useful in the invention. For example, such antibodies can be used in an immunoassay to monitor the amount of MAP44 produced by an animal (for example, to determine the level in body fluids, the tissues, cellular or subcellular location of MAP44).

Preferably, antibodies of the invention are produced using fragments of the MAP44 protein, which lie outside highly conserved regions and appear likely to be antigenic, by criteria such as high frequency of charged residues. In one specific example, such fragments are generated by standard techniques of PCR, and are then cloned into the pGEX expression vector (Ausubel et al., supra). Fusion proteins are expressed in E. coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two rabbits. Antisera can be raised by injections in a series, preferably including at least three booster injections.

Antiserum is also checked for its ability to immunoprecipitate recombinant MAP44 proteins or control proteins, such as glucocorticoid receptor, CAT, or luciferase.

The antibodies can be used, for example, in the detection of the MAP44 in a biological sample as part of a diagnostic assay. Antibodies also can be used in a screening assay to measure the effect of a candidate compound on expression or localization of MAP44. Thus, the antibody may be coupled to a compound comprising a detectable marker for diagnostic purposes. Additionally, such antibodies can be used in conjunction with the gene therapy techniques described to, for example, evaluate the normal and/or engineered MAP44-expressing cells prior to their introduction into the patient. Such antibodies additionally can be used in a method for inhibiting abnormal MAP44 activity.

In addition, techniques developed for the production of “chimeric antibodies” (Morrison et al., Proc. Natl. Acad. Sci. USA, 81:6851, 1984; Neuberger et al., Nature, 312:604, 1984; Takeda et al., Nature, 314:452, 1984) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. Nos. 4,946,778, 4,946,778, and 4,704,692) can be adapted to produce single chain antibodies against a MASP-2 protein or polypeptide. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂ fragments. Alternatively, Fab expression libraries can be constructed (Huse et al., Science, 246:1275, 1989) to allow rapid and easy identification of monoclonal Fab fragments with the desired specificity.

Antibodies to MAP44 can, in turn, be used to generate anti-idiotype antibodies that resemble a portion of MAP44 using techniques well known to those skilled in the art (see, e.g., Greenspan et al., FASEB J. 7:437, 1993; Nissinoff, J. Immunol. 147:2429, 1991). For example, antibodies that bind to MAP44 and competitively inhibit the binding of a ligand of MAP44 can be used to generate anti-idiotypes that resemble a ligand binding domain of MAP44 and, therefore, bind and neutralize a ligand of MAP44 such as MBL or ficolins. Such neutralizing anti-idiotypic antibodies or Fab fragments of such anti-idiotypic antibodies can be used in therapeutic regimens.

Antibodies can be humanized by methods known in the art. For example, monoclonal antibodies with a desired binding specificity can be commercially humanized (Scotgene, Scotland; Oxford Molecular, Palo Alto, Calif.). Fully human antibodies, such as those expressed in transgenic animals are also features of the invention (Green et al., Nature Genetics 7:13-21, 1994; see also U.S. Pat. Nos. 5,545,806 and 5,569,825, both of which are hereby incorporated by reference).

The methods described herein in which anti-MAP44 antibodies are employed may be performed, for example, by utilizing pre-packaged diagnostic kits comprising at least one specific MAP44 nucleotide sequence or antibody reagent described herein, which may be conveniently used, for example, in clinical settings, to diagnose patients exhibiting symptoms of the disorders described below.

### Quantitative Assays of MAP44

As an example only, quantitative assays may be devised for the estimation of MAP44 concentrations in body fluids or organ (biopsy) extracts. Such assays may be fluid phase or solid phase. Examples are competitive and non-competitive ELISAs. As an example of the latter, microtiter wells are coated with anti-MAP44 anti-body, incubated with samples, and the presence of MAP44 visualized with enzyme-labelled antibody followed by substrate that is cleaved into a colored compound.

Alternatively, a label such as europium may be used and the detection made by use of time resolved fluorometry.

Assays of the functional activity of MAP44, either alone or as part of the MBL/MASP complex may be performed by several methods. The activity of MAP44 to inhibit the C4 cleaving effect of MBL/MASP-2 complex may be assayed by the following method, comprising the steps of

applying a sample comprising MBL/MASP-2 complexes to a solid phase obtaining bound complexes

applying MAP44 to the bound complexes

applying at least one complement factor to the complexes

detecting the amount of cleaved complement factors

correlating the amount of cleaved complement factors to the MAP44 activity

The solid phase may be any coating capable of binding MBL, such as a mannan coating or any coating capable of binding ficolins, such as a surface of acetylated albumin, or a specific antibody against MBL or the ficolins.

The complement factor preferably used in the present method is a complement factor cleavable by the MBL/MASP-2 complex, such as C4. However, the complement factor may also be selected from C3 and C5 or the soluble terminal C complex, sTCC.

The cleaved complement factor may be detected by a variety of means, such as by of antibodies directed to the cleaved complement factor.

Assays estimating the activity of MAP44 or quantity of MAP44 may be used for diagnostic and treatment purposes in samples from individuals, notably those suffering from infectious or inflammatory diseases.

### MAP44 for Therapy

Therapeutic use of components specified in the claims may be applied in situations where a constitutional or temporary deficiency in MAP44 renders the individual susceptible to activation of the complement system. MAP44 can be administered, preferably by intravenous infusions, in order to stabilize the individual's inflammatory reactions or modulate autoimmune manifestations. Recombinant MAP44 may be in the form of the whole molecule, parts of the molecule, or the whole or part thereof attached by any means to another structure in order to modulate the activity or the half-life in the body. The recombinant products may be identical in structure to the natural molecule or slightly modified to yield enhanced activity or decreased activity when such is desired.

Conditions to be treated are not limited to presently known conditions for which there exists a need for treatment. The conditions comprise generally any condition in connection with current and/or expected need or in connection with an improvement of a normal condition. In another aspect of the present invention the manufacture is provided of a medicament comprising a pharmaceutical composition comprising functional MAP44, or any variant thereof, intended for treatment of conditions comprising cure and/or prophylaxis of conditions of diseases and disorders of, e.g., the immune system and reproductive system by humans and by animals having said functional units acting like those in humans.

Thus, in particular the pharmaceutical composition comprising MAP44 or a functional variant thereof may be used for the treatment and/or prevention of clinical conditions selected from infections, cancer, disorders associated with chemotherapy, such as infections, diseases associated with human immunodeficiency virus (HIV), diseases related with congenital or acquired immunodeficiency. More particularly, chronic inflammatory demyelinating polyneuropathy (CIDP, Multifocal motoric neuropathy, Multiple scelrosis, Myasthenia Gravis, Eaton-Lambert's syndrome, Opticus Neuritis, Epilepsy; Primary antiphosholipid syndrome; Rheumatoid arthritis, Systemic Lupus erythematosus, Systemic scleroderma, Vasculitis, Wegner's granulomatosis, Sjøgren's syndrome, Juvenile rheumatiod arthritis; Autoimmune neutropenia, Autoimmune haemolytic anaemia, Neutropenia; Crohn's disease, Colitis ulcerous, Coeliac disease; Asthma, Septic shock syndrome, Chronic fatigue syndrome, Psoriasis, Toxic shock syndrome, Diabetes, Sinusitis, Dilated cardiomyopathy, Endocarditis, Atherosclerosis, Primary hypo/agammaglobulinaemia including common variable immunodeficiency, Wiskot-Aldrich syndrome and severe combined immunodefiency (SCID), Secondary hypo/agammaglobulinaemia in patients with chronic lymphatic leukaemia (CLL) and multiple myeloma, Acute and chronic idiopathic thrombocytopenic purpura (ITP), Allogenic bone marrow transplantation (BTM), Kawasaki's disease, and Guillan-Barre's syndrome. Also before, during or after reestablishment of blood flow following a blocade, e.g., ischemia/reperfusion, or other conditions associated with decreased blood flow.

The route of administration may be any suitable route, such as intravenously, intramusculary, subcutanously or intradermally. Also, pulmonal or topical administration is envisaged by the present invention. Oral administration must also be considered when a suitable formulation allowing uptake from the intestine is developed.

An examination of the biological activity of MAP44 carried out by using recombinant proteins produced in a mammalian expression system revealed a pronounced inhibitory activity of rMAP44 on the activation of C4 by natural MBL complexes. The activity of rMBL-rMASP-2 complexes was also inhibited by rMAP44.

There is accordingly provided a method for inhibiting complement activation by inhibiting the lectin pathway, said method comprising the step of administering an effective amount of MAP44, or a functional variant thereof, to an individual in need of complement down-regulation and/or complement inhibition.

In one preferred embodiment of the present invention there is provided a method for inhibiting the activation of C4 complement by inhibiting the lectin pathway, said method comprising the step of administering an effective amount of MAP44 or a functional variant thereof to an individual in need of C4 down-regulation and/or C4 inhibition.

There is also provided a method for inhibiting MASP-2 activity, said method comprising the step of administering an effective amount of MAP44, or a functional variant thereof, to an individual in need of MASP-2 down-regulation and/or MASP-2 inhibition. In one presently preferred embodiment MAP44 is capable of inhibiting complex formation between MASP-2 and MBL or ficolins.

In a still further embodiment there is provided a method for inhibiting or treating an inflammatory condition in an individual, in particular a condition related to complement activation through MBL/MASP or ficolin/MASP complexes, said method comprising the step of administering an effective amount of MAP44, or a functional variant thereof, to an individual in need of treatment for an inflammation. The inflammatory condition may be chronic, such as, e.g., rheumatoid arthritis or systemic lupus erythematosus, or the inflammatory condition may be an acute inflammatory condition. The treatment according to the invention is in one such embodiment directed against treatment of reoxygenated ischemic tissues. The inflammatory condition may also result from an autoimmune condition.

In a still further embodiment there is provided a method for treating an individual suffering from a disorder resulting from an imbalanced cytokine network, e.g., a disorder involving or resulting from an unfavourable TNF response to bacterial lipopolysaccharides, said method comprising the step of administering an effective amount of MAP44, or a functional variant thereof, to an individual in need thereof.

### Use of MAP44 for Clinical Purposes

The polypeptide according to the invention may be used for a variety of clinical purposes, such as for administration as a pharmaceutical composition. Thus, in one aspect the present invention relates to the use of the polypeptide according to the invention, or a compound as defined herein for preparation of a pharmaceutical composition.

The pharmaceutical composition is preferably capable of being administered parenterally, such as intramusculary, intravenously, or subcutaneously, or capable of being administered orally.

As discussed above with respect to therapy with MAP44 the pharmaceutical composition may be used for a wide variety of diseases and condition, such as the treatment of MAP44 deficiency.

### Assays for MAP44

Therapy with MAP44 (or MAP44 inhibitors) must usually be preceded by the estimation of MAP44 in serum or plasma from the patient. Examples of such assays are described below.

### Assays for MAP44 Antigen

MAP44 protein is conveniently estimated as antigen using one of the standard immunological procedures. Thus, the invention relates to a method for detecting mannan-binding lectin associated protein (MAP44) in a biological sample, said method comprising:

(a) obtaining a biological sample;

(b) contacting said biological sample with a MAP44 polypeptide specific binding partner that specifically binds MAP44; and

(c) detecting said complexes, if any, as an indication of the presence of MAP44 in said sample.

The binding partner may be any molecule capable of selectively binding to MAP44 and capable of being detectable, such as by labelling with a detectable label. The specific binding partner may thus be an antibody as described herein, or a mannan-binding lectin (MBL) or any of the ficolins.

As an example only, a quantitative TRIFMA (time resolved immunofluorometric assay) for MAP44 was constructed by 1) coating microtitre wells with 1 μg antibody reacting with MAP44; 2) blocking with Tween-20; 3) applying test samples, e.g., diluted plasma or serum samples; 4) applying biotin labelled anti-MAP44 antibody; 5) applying Eu-labelled streptavidin; 6) applying enhancement solution (Perkin Elmer Ltd); 7) reading the Eu on a time resolved fluorometer (Estimation by ELISA (enzyme linked immunosorbent assay) may be carried out similarly, e.g., by using biotin-labelled anti-MAP44 in step 4; enzyme-labelled avidin in step 5; 6) apply substrate; and 7) read the colour intensity). Between each step, the plate is incubated at room temperature and washed, except between step 6 and 7. A calibration curve may be constructed using dilutions of pooled normal plasma, arbitrarily said to contain 1 unit of MAP44 per ml or normal plasma with a known concentration of MAP44. Alternatively, the secondary antibody may be directly labelled with enzyme or with europium or with a fluorescent molecule.

Assays may be similarly constructed using antibodies, polyclonal or monoclonal or recombinant antibodies, which reacts with MAP44, natural or recombinant, or parts thereof.

Through the use of antibodies reacting selectively with intact MAP44 or with activation products, or through combination of antibodies against various parts of the molecule, assays may be constructed for the estimation of the activation in vivo of the lectin pathway. These assays will be useful for the determination of inflammation caused by the activation of this pathway.

One may assay for the total amount of MAP44 in a sample as outlined above. Alternatively one may assay for MAP44 present in complexes or assay only for free MAP44. To do this one may separate complexes from free MAP44 by any suitable method well known to skilled artisans, e.g., by treatment of the sample with a precipitating agent or by size separation by means of filtration or by removal of MBL or ficolins with antibodies.

One may choose to estimate specifically the amount of MAP44 bound to MBL or one of the ficolins. The assay may be carried out as described in the following manner:

The assay carried out in the TRIFMA formate proceeds as follows: 1) coating microtitre wells with anti-MBL or anti ficolin in 100 μl buffer; 2) blocking with Tween-20; 3) incubate with 100 μl of diluted sample; 4) wash and applying anti-MAP44 antibody labelled with biotin; 6) applying Eu-labelled streptavidin; 7) applying enhancement solution; and 8) reading the Eu by time resolved fluorometry (Estimation by ELISA may be carried out similarly, e.g., by applying enzyme-labelled streptavidin; 8) apply substrate; and 9) read the colour intensity). A calibration curve may be constructed using dilutions of one selected plasma pool, arbitrarily said to contain 1 unit of MAP44 complex per ml.

### EXAMPLES

### Example 1

### Identification of MAP44

A putative novel mRNA product of the MASP1 gene was identified in NCBI's gene database as AL134380.1 and BC039724.1, the former was a 621 bp mRNA fragment (Blum et al. 1999, unpublished), and the latter was a 2065 bp mRNA(37). The putative protein product encompasses CUB1-EGF-CUB2-CCP1 (363 amino acids) of MASP-1/-3 and additional 17 unique amino acids (KNEIDLESELKSEQVTE (SEQ ID NO 4) C-terminally. The calculated MW of the polypeptide product was 44 kDa, and we have named this candidate protein “mannan-binding lectin-associated protein of 44 kDa”, or “MAP44”.

As the clones described above were derived from human fetal brain we searched for the transcript using a MAP44-specific primer set in PCR reactions on human brain cDNA and cDNA from various brain-derived cell-lines, as well as HeLa and HEK293 cells. PCR on human brain cDNA yielded a band of the expected size for specific MAP44 amplification (FIG. S1A). Sequencing this product confirmed its identity with the expected region of MAP44 mRNA. This product was also seen, albeit weaker, with NT2 cells, and even weaker with A172, NHA, and HeLa cells. All of these cells also gave a product with a common MASP1 gene expression primer set (FIG. S1B).

### Example 2

### Features of the Gene, Splicing, and the Resulting mRNA

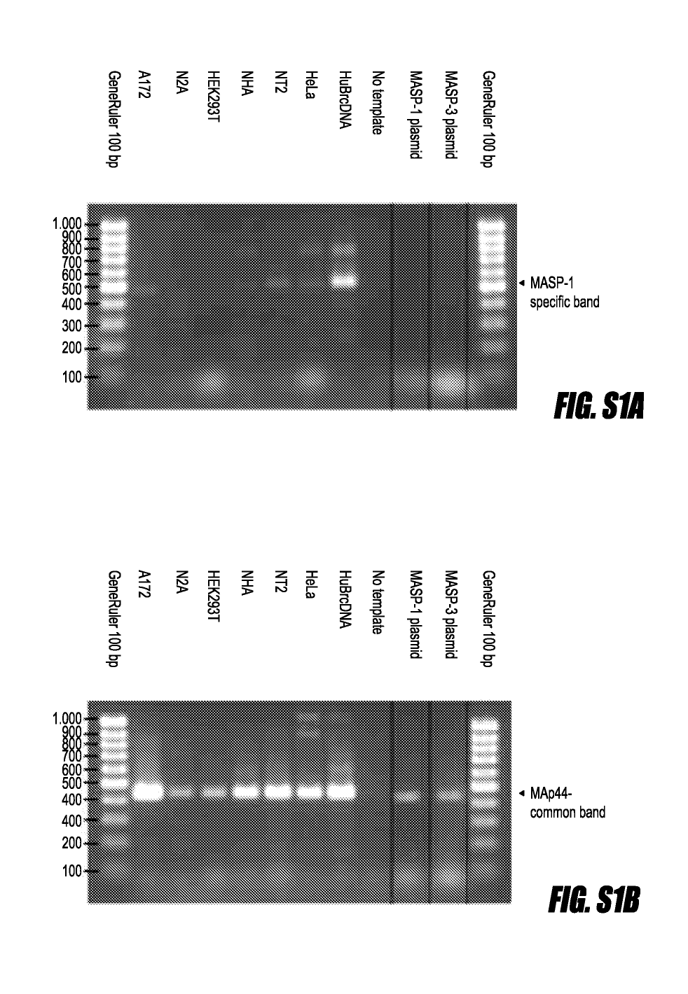

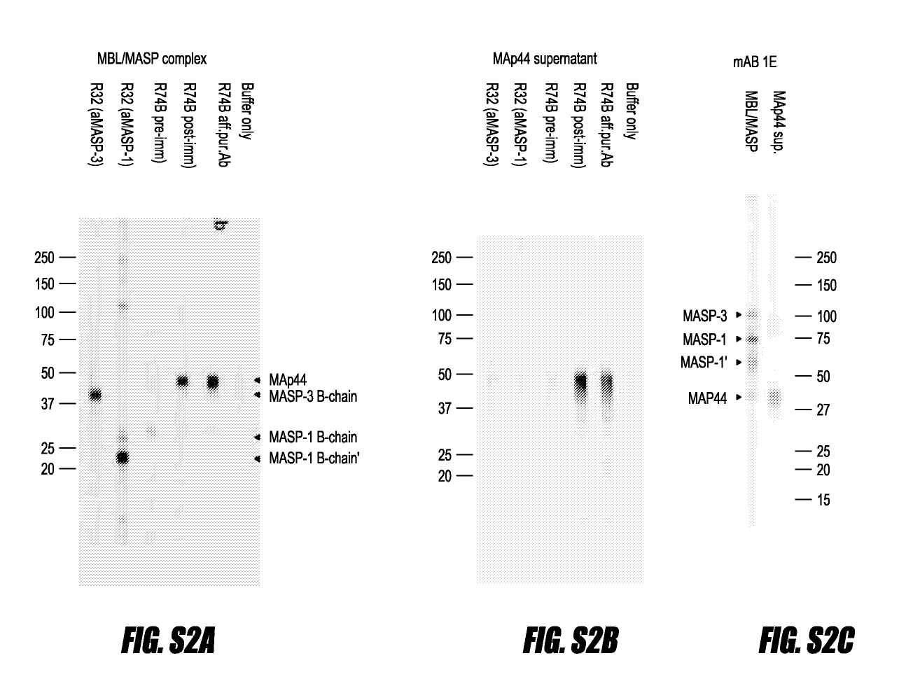

The MAP44 splice product is produced from 9 exons: the first 8 exons are shared with the MASP-1 and MASP-3 splice products and code for the CUB1, EGF, CUB2 and CCP1 domains, whereas the 9th exon is unique to MAP44. An additional adenosine nucleotide from exon 8 combined with the first 50 nucleotides of exon 9 code for the 17 unique amino acids of MAP44 (FIGS. 1, A and B). Exon 9 also contains an extensive 3′UTR, which houses the polyA signal.

The splice donor site of exon 8 and splice acceptor site of exon 10 of MASP-1 and -3 are highly similar to the consensus sequences (MAGGTRAGT, M=A/C (SEQ ID NO 5), R=A/G and YYYYYYYYYYYNYAG, Y=C/T, (SEQ ID NO 6) respectively) (FIG. 1B). The acceptor site of exon 9 is less conserved, although presenting the crucial terminal AG. Both splice events conform to the GT/AG rule(38), but only the intron 9/exon 10 junction presents a canonical polypyrimidine tract.

A conventional polyA site is absent in MAP44 mRNA. However, PolyApred (Ahmed, F., Kumar, M., and Raghava, G. P. S., unpublished) predicts a putative novel polyA signal with the sequence CCAGAC (SEQ ID NO 7) starting at position 1881. The mRNA was shown by sequencing to have a 3′-terminal poly(A) sequence starting at position 1990.

### Example 3

### mRNA Levels in Human Tissues

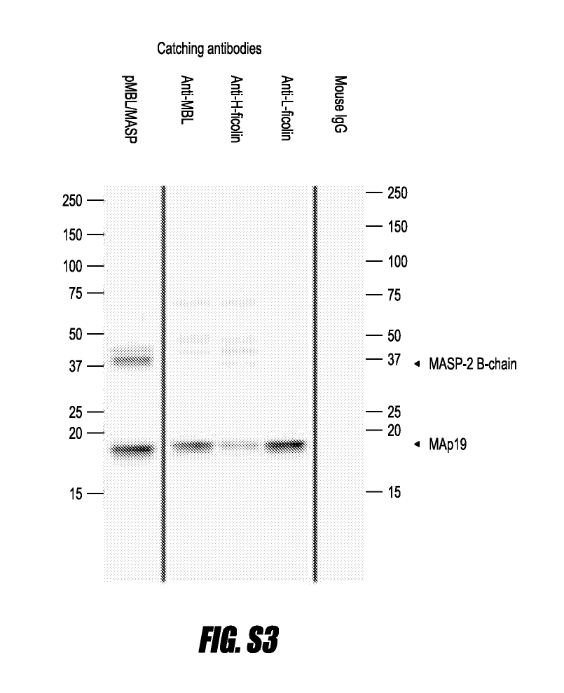

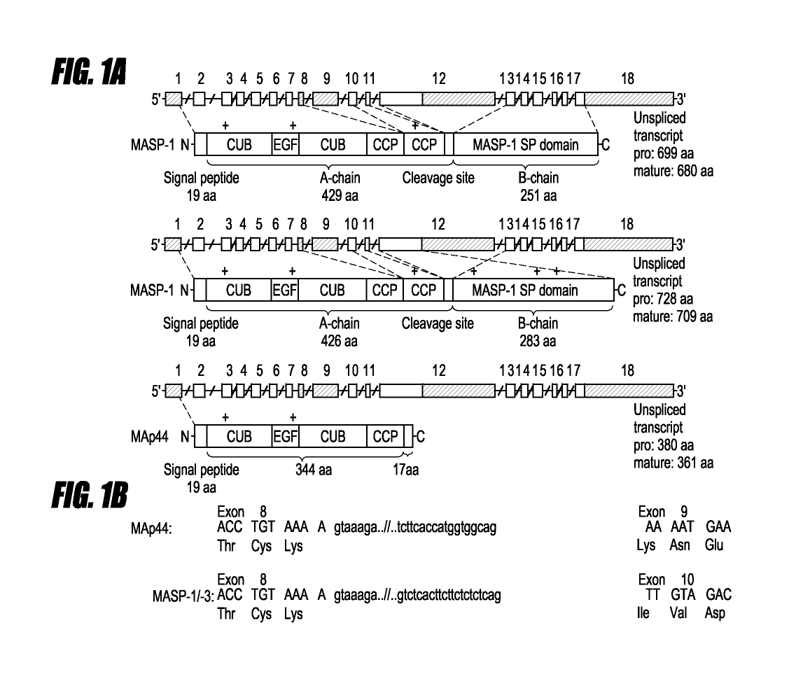

The levels of mRNA encoding MAP44, MASP-1 and MASP-3 in a tissue library were compared with qRT-PCR using beta₂-microglobulin mRNA levels for normalization. The site of highest relative expression level of MAP44 was the heart followed by much weaker expression in liver, brain and cervix (FIG. 2A). Apart from the heart the expression profile of MAP44 is similar to that of MASP-3 (FIG. 2B). MASP-1 mRNA, on the other hand, is predominantly found in liver tissue, with only low copy numbers in cervix, brain, placenta, prostate and bladder (FIG. 2C).

### Example 4

### Identification of MAp44 in Complex with MBL and Ficolins in Human Se-Rum

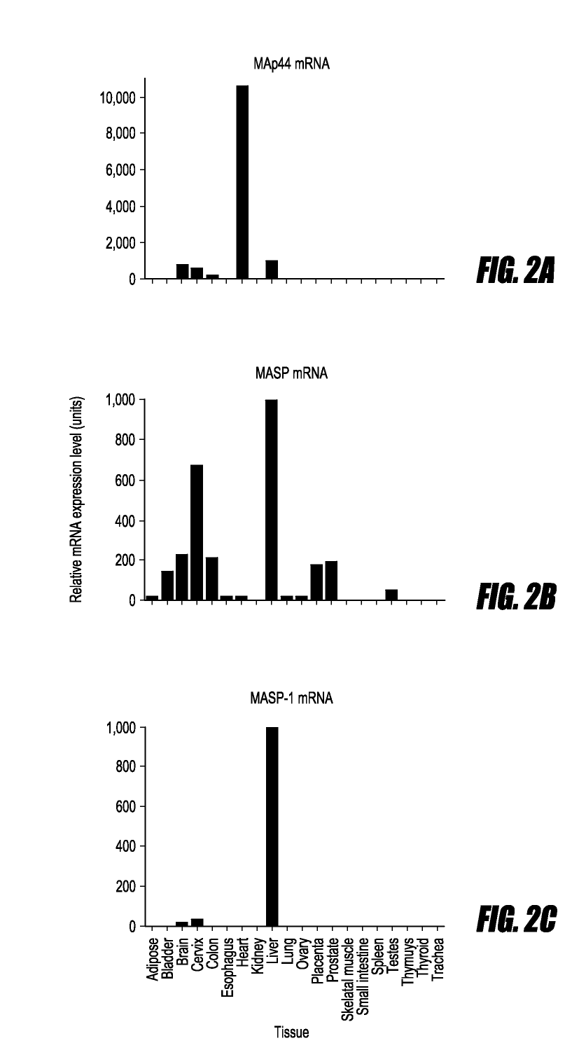

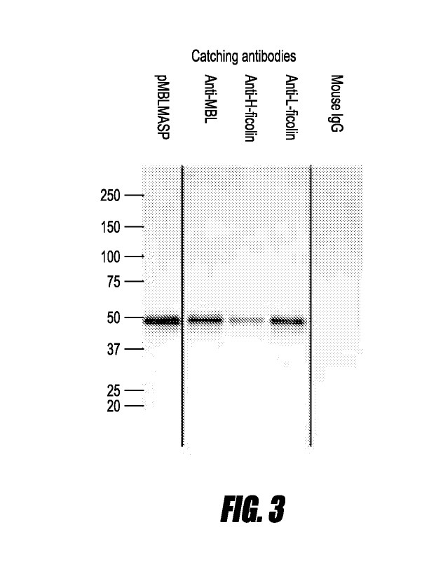

To study MAp44 at the protein level we purified MBL/MASP complexes from human plasma, we produced rMAp44 in a human cell line, and we raised polyclonal rabbit anti-MAp44 antibody (pAb) using a peptide representing the C-terminal 19 amino acids of MAp44. Antiserum and the affinity-purified antibody generated a single band of the expected size of 44 kDa when tested on blots of purified MBL/MASP complex (FIG. S2A) and rMAp44-containing supernatant (FIG. S2B). The MAp44 band was also seen when developing with monoclonal antibody (mAb) 1E2 (recognizing an epitope in the common N-terminal of MASP-1/-3/MAp44) (FIG. S2C). To search for the presence of MAp44 in complexes with MBL or ficolins we used antibody-coated micro-wells to affinity purify complexes from serum, which were then analyzed by Western blotting. Bands at the position expected for MAp44 were seen in the lanes containing the eluate from wells coated with anti-MBL, anti-H-ficolin and anti-L-ficolin (FIG. 3A), as well as in the lane with directly loaded MBL/MASP complexes purified conventionally from serum. In separate experiments we developed identical blots with mAb anti-MASP-2/MAp19 (mAb 1.3B7) to confirm capture of complexes (FIG. S3) and blots of MBL/MASP complexes with mAb 1.3B7 (FIG. S3), mAb 1E2, pAb anti-MASP-1, and pAb anti-MASP-3 (FIGS. S2, A and C), to confirm the positions of MAp19, MASP-2, MASP-1 and MASP-3 relative to MAp44.

In addition, we similarly captured MBL and ficolins from serum and probed in situ with anti-MAp44 or anti-MASP-3 antibodies. We observed dose dependent signals in wells coated with anti-MBL, anti-H-ficolin and anti-L-ficolin but not in wells coated with mouse IgG (FIGS. 3, B and C). As a positive control we included wells coated with mAb 1E2. We conclude that MAp44 is associated with MBL, H-, and L-ficolin in human serum.

### Example 5

### Quantification of MAp44 in Human Serum

We constructed a solid-phase assay for the quantification of MAp44. Microtiter wells were coated with mAb 1E2, incubated with samples, and developed with biotinylated rabbit anti-MAp44. The samples were diluted in a buffer containing EDTA and high salt, ensuring the dissociation of sPRM/MASP/MAp complexes. The MAp44 content was estimated by comparison with highly purified rMAp44. The mean concentrations in serum and EDTA plasma from 74 blood donors were 1.38 μg/ml (range 0.34-3.00 μg/ml) and 0.80 μg/ml (range 0.14-2.04 μg/ml), respectively. The distribution of MAp44 conformed to a normal log distribution.

### Example 6

### Surface Plasmon Resonance Analysis of the Interaction Between MAp44 and MBL

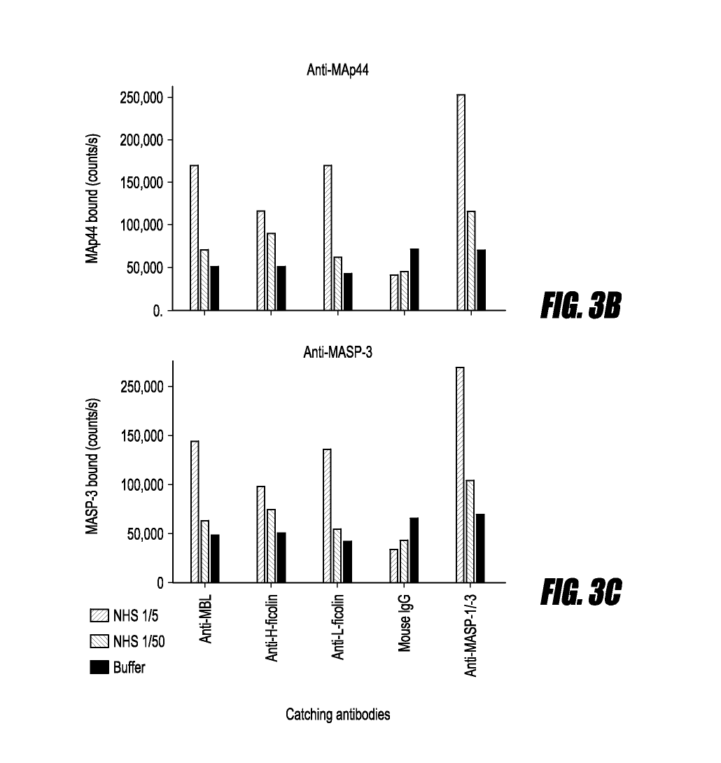

Using SPR we determined the strength of the interaction between MAp44 and MBL, and compared it to that of MASP-3 and MBL. The purity of the rMBL has been reported before and the rMAp44 and rMASP-3 preparations were deemed pure by silver staining of SDS-PAGE gels (FIG. 4A). MBL was coupled to SPR chips at two different densities. An SPR chip, activated and blocked, was used for subtraction of the bulk refractive index background. A BSA-coated surface served as an extra background control, which gave no higher signal than the blank surface for both MASP-3 and MAp44. Representative sensorgrams are shown for MAp44 binding and MASP-3 binding (FIGS. 4, B and C), yielding K_{D}s of 0.6 nM (kₐ of 1.34×10⁵ s⁻¹M⁻¹, k_{d} of 7.82×10⁻⁵ s⁻¹, Chi² of 4.6) and 0.4 nM (kₐ of 9.3×10⁴ s¹M⁻¹, k_{d} of 3.77×10⁻⁵ s⁻¹, Chi² of 30), respectively. The measurements at the other coupling density of MBL were in agreement for both MASP-3 and MAp44. The calculated K_{D}s were similar to the 0.8 nM reported for the binding of MASP-2 to MBL.

### Example 7

### The Size Distribution of MAp44 in Serum

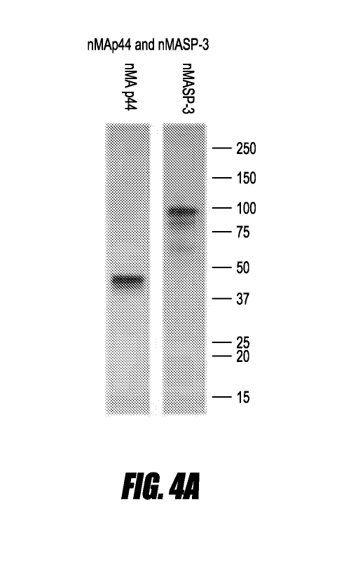

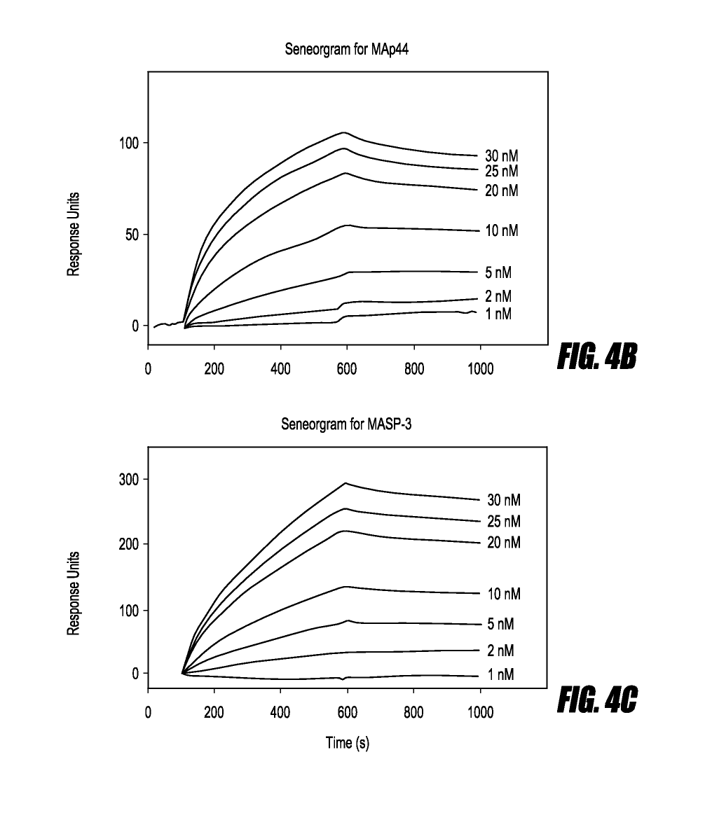

NHS was subjected to GPC in an isotonic, Ca²⁺-containing buffer, or in a buffer containing EDTA and a high salt concentration (dissociating conditions). MAp44 was found to elute as closely overlapping twin peaks at around 11 and 12 ml in the Ca²⁺-containing buffer (FIG. 5). Under dissociating conditions a single, symmetrical peak was seen at 14.5 ml, corresponding to an apparent MW of around 180 kDa. This profile suggests that MAp44 is found in high molecular weight complexes with MBL and ficolins, and that these complexes are dissociated under high salt+EDTA conditions. These findings compare well with those reported for the MASPs and MAp19(21). A similar GPC analysis of purified rMAp44 gave a peak corresponding to MAp44 in serum under dissociating conditions.

### Example 8

### Competition Between MAp44 and MASP-3 in Binding to MBL

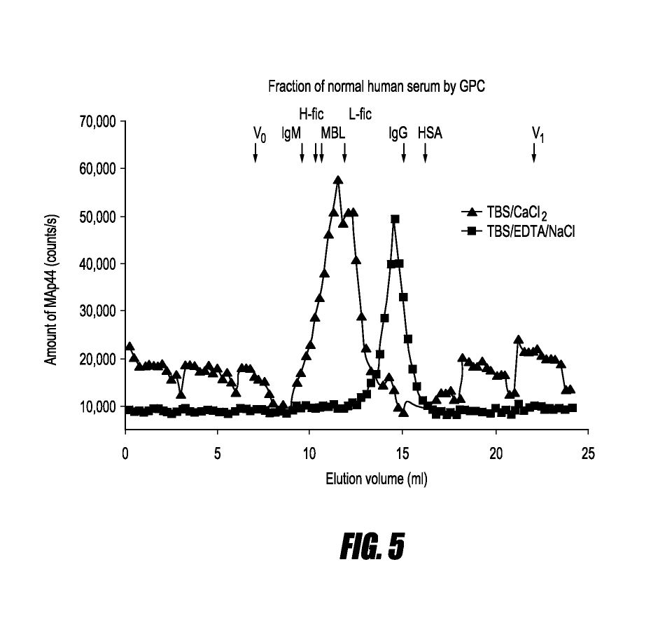

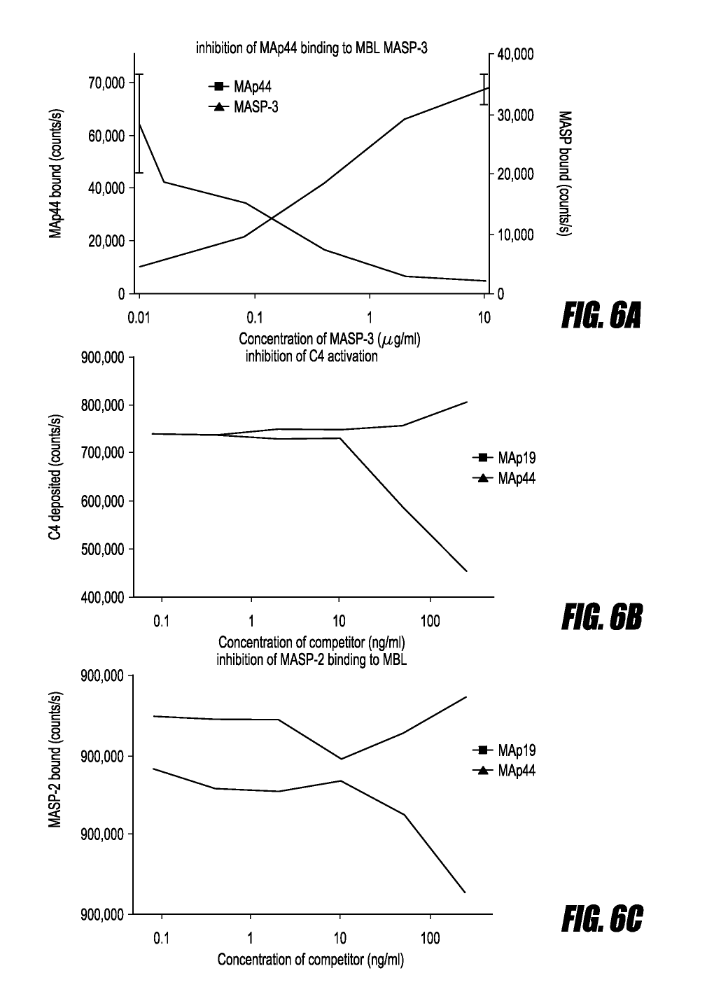

We assayed the ability of MAp44 to compete with MASP-3 for binding to MBL. Complexes with MBL were formed in solution and the mixtures added to mannan-coated wells to allow MBL to bind. The wells were washed and developed with either anti-MAp44 or anti-MASP-3 antibodies. When MAp44 and MASP-3 where incubated simultaneously with MBL, competition between the two in binding to MBL was observed (FIG. 6A). We conclude that MAp44 and MASP-3 bind to the same or over-lapping sites on MBL.

### Example 9

### MAp44 Competes with MASP-2 for Binding to MBL and Down-Regulates C4 Cleavage

MASP-2, the C4 activating component of the sPRM/MASP complexes harbors MBL-binding domains that are not identical to those of MASP-1, MASP-3 and MAp44, but have a similar configuration. It seemed possible that MAp44 might compete with MASP-2 for binding to MBL. Since such a role was also suggested for MAp19 this protein was included in our examinations. We incubated MBL with MAp44 or MAp19 at various concentrations, followed by incubation with MASP-2. The complexes were allowed to bind to a mannan-coated surface, followed by incubation with C4, and finally detection of deposited C4 fragments. MAp44 inhibited C4 deposition, while MAp19 did not (FIG. 6B). These observations may be explained by the high affinity for MBL of MAp44, which is very similar to that of MASP-2, whereas that of MAp19 is more than 10-fold lower (around 13 nM).

We also measured the amount of bound MASP-2 and bound competitor in the complexes in situ. The amount of bound MASP-2 was decreased when adding MAp44 but not when adding MAp19 (FIG. 6C). We conclude that MAp44 competes with MASP-2 for binding to MBL, resulting in inhibition of C4 deposition, and hence inhibition of downstream complement activation.

### Example 10

### Phylogenetics

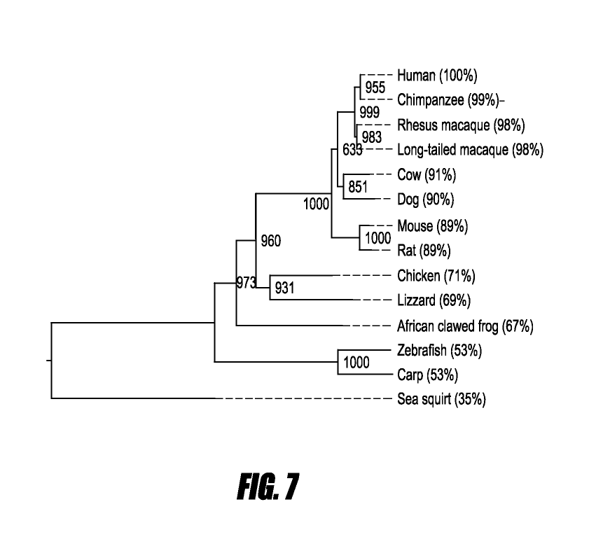

A database search identified orthologs of MAp44 in mammals (chimpanzee, macaque, dog, mouse, and rat) as well as in bony fish (carp and zebrafish). The carp orthologue has been described in the literature at the transcript level as MRP(29). A homologue of MRP has been described in sea squirt (a urochordate) at the genomic level(30, 31). This prompted us to conduct further database studies as delineated in Materials and Methods. MAp44 was absent in Branchiostoma and present in Xenopus, chicken and lizzard, as well as cow. Its presence/absence could not be determined in shark and lamprey, due to the incompleteness of their genomes. The results are compiled in Table SI, and the resulting phylogenetic tree is shown in FIG. 7. Although it is quite well conserved, the hallmark feature of MAp44, i.e., the C-terminal tail, differs radically between fish and mammals.

### Materials and Methods

### Analysis of Gene Structure

The gene was analyzed using the programs Human Splicing Finder, v. 2.3 (Hamroun, D., Desmet, F. O., and Lalande, M., unpublished), polyadq(1), DNA functional site miner—Poly(A) Signal Miner(2), and PolyApred (Ahmed, F., Kumar, M., and Raghava, G. P. S., unpublished).

### RT-PCR and Sequencing

Primers were designed to amplify a 497 bp fragment from MAp44 mRNA (forward primer in exon 8, reverse primer in the 3′UTR of the unique exon 9). PCR was per-formed on cDNA made from cell line and tissue RNA (3). The product arising from PCR on human brain cDNA was purified and sequenced.

Quantitative Real-Time Reverse Transcriptase-Polymerase Chain Reaction (qRT-PCR)

mRNA expression levels were quantified in a FirstChoice Human Total RNA Survey Panel (Applied Biosystems®/Ambion®) comprising RNA from 20 human tissues, employing TaqMan® chemistry and the ABI Prism 7000 Sequence Detection System. The RNA was reverse transcribed using the Roche® One Step RT-PCR system with oligo-dT primers. TaqMan® gene expression assays from Applied Biosystems® were used for MASP-1 (cat. no. Hs01111256_m1), MASP-3 (Hs01111266_m1), and MAp44 (Hs01112777_m1), using β₂m mRNA (Hs99999907_m1) for normalization. The relative levels of MASP-1, MASP-3, and MAp44 mRNA were compared using the delta-delta Cₜ method.

### Anti-MAp44 Antibody

The C-terminal 19 amino acids of MAp44 contain the 17 unique C-terminal amino acids as well as an N-terminal cysteine for MBS-coupling to keyhole limpet hemocyanin. Two rabbit antisera, R74A and R74B, were obtained after immunization regimes, and their antibodies affinity purified on peptide-coupled Sepharose 4B beads. These procedures were carried out by GenScript.

The antibodies were tested on Western blot strips of purified MBL/MASP complexes (containing 30 μg MBL, resulting in approximately 1 μg MBL per strip) or rMAp44 supernatant (containing 300 μl supernatant, 10 μl per strip) run on single-well XT-Criterion 4-12% gradient Bis-Tris polyacrylamide gel (Bio-Rad) using XT-MOPS running buffer (Bio-Rad®) either reduced or non-reduced as indicated. Precision All Blue pre-stained marker (Bio-Rad®) was used for the estimation of molecular sizes. The proteins in the gel were blotted to Hybond-ECL membrane (GE Healthcare®) in transfer buffer (25 mM Tris, 0.192 M glycine, 20% v/v ethanol, 0.1% w/v SDS, pH 8.3), the membrane was blocked in 0.1% Tween in TBS, and then cut into 2.5 mm wide strips, which were incubated in the wells of Octaline trays (Pateof) with primary antibodies primary buffer (TBS/Tw, 1 mM EDTA, 1 mg HSA/ml, 100 μg normal hu-man IgG (hIgG)/ml). The strips were washed, incubated with secondary antibody in secondary buffer (TBS/Tw, no azide, 1 mM EDTA, 100 μg hIgG/ml), and washed again before being developed with SuperSignal West Dura Extended Duration Substrate (Pierce®). Images were taken using a CCD camera (LAS-3000, Fuji) and analyzed with the MultiGauge Image Analysis Software supplied with the camera.

The primary antibodies used for Western blotting were R74A and R74B rabbit anti-MAp44 antisera, pre-immune sera, as well as the affinity-purified R74A and R74B antibodies, mouse monoclonal anti-MASP-1/MASP-3/MAp44 common determinant (1E2, Hycult Biotechnology, HBT), polyclonal rabbit anti-MASP-3 (R32) and polyclonal rabbit anti-MASP-1 (R64). The secondary antibodies were HRP-conjugated goat anti-rabbit IgG (Dako®) and HRP-conjugated rabbit anti-mouse Ig (Dako®).

### Recombinant Proteins

Recombinant MBL (rMBL) was produced as described (4). MBL/MASP complexes were purified from human plasma. MAp44 cDNA in the vector pCMV-SPORT6 was purchased from imaGenes (clone IRAKp961F1682Q) and the insert sequenced. Plasmids encoding MASP-3 and MAp19 have been described. rMAp44, rMASP-3 and rMAp19 were produced by transient expression in 293F cells (Invitrogen®) and purified by affinity chromatography on rMBL-coupled beads by binding in a Ca²⁺-containing buffer and eluting in a buffer containing EDTA and 1 M NaCl. The purity was verified by silver staining of SDS-PAGE gels, and the concentrations were determined by OD measurement and quantitative amino acid analysis.

### Assay of MAp44

A sandwich assay was developed, involving capture with mAb 1E2 (reacting with the N-terminal domains shared by MASP-1, MASP-3 and MAp44) and detection of bound MAp44 with biotinylated anti-MAp44 antibody followed by Eu³⁺-labeled streptavidin. The amount of Eu³⁺ in the wells was read by time-resolved fluorometry (TRIFMA).

MAp44 Associated with MBL and Ficolins in Serum

MBL- or ficolin-containing complexes were extracted by microtiter well based affinity chromatography. Wells were coated with 131-1 (mAb anti-MBL, Bioporta), 4H5 (mAb anti-H-ficolin, HBT), GN5 (mAb anti-L-ficolin, HBT) or monoclonal non-specific mouse IgG₁ (Sigma®), then incubated with diluted normal human serum (NHS), washed, and bound material eluted with SDS-PAGE sample buffer. The samples were analyzed by Western blotting using rabbit anti-MAp44 antibodies. MAp44 in complex with MBL or ficolins was also analyzed by TRIFMA. Wells were coated with 131-1, 4H5, GN5, 1E2, or non-specific mouse IgG₁, incubated with diluted NHS, washed, added rabbit anti-MAp44 or anti-MASP-3 followed by biotinylated swine anti-rabbit Ig, and development with Eu³⁺-labeled streptavidin. Finally, we also confirmed capture of complexes by developing in a similar set-up using mouse monoclonal anti-MASP-2/MAp19 antibody (1.3B7) (5).

### Gel Permeation Chromatography (GPC)

NHS or rMAp44 were subjected to GPC on a Superose 6 column in either Ca²⁺-containing buffer or high salt+EDTA buffer(6). MAp44 was quantified in the fractions as described above. Fractions were also analyzed for IgM, MBL and H-ficolin.

### Surface Plasmon Resonance (SPR)

The SPR experiments were similar to those reported (7, 8). Using a BIAcore 3000 instrument (GE Healthcare®), binding of either rMASP-3 or rMAp44 was measured on 10940 RU of rMBL immobilized on a CM5 sensor chip (30 μg rMBL/ml used for derivatization), at a flow rate of 5 μl/min. Equivalent volumes of rMASP-3 and rMAp44 were injected at concentrations from 1 nM to 30 nM. Data were analyzed by global fitting to a 1:1 Langmuir binding model for several concentrations simultaneously using the BIAevaluation 4.1 software (GE Healthcare®).

### Competitive Binding to MBL

Fixed concentrations of rMAp44 and rMBL were incubated with increasing concentrations of rMASP-3. The mixtures were then incubated in mannan-coated microtiter wells. After incubation and washing, the wells were incubated with biotin-labeled pAb against MAp44 or mAb against MASP-3 (mAb 38.12-3) and developed with Eu³⁺-labeled streptavidin.

### Effect of MAp44 on Activation of the Lectin Pathway

Dilutions of rMAp44 or rMAp19 were made in 10 mM Tris-HCl, 1 M NaCl, 5 mM CaCl₂, 100 μg HSA/ml, 0.05% Triton X-100, pH 7.4 (binding buffer) and rMBL was added to reach 50 ng MBL/ml. A preparation of rMASP-2 (9) was diluted to 5 ng/ml in binding buffer and added to an equal volume of the mixtures above (reaching a final concentration of 25 ng rMBL/ml, 2.5 ng rMASP-2/ml and varying amounts of MAp44 or MAp19). The mixtures were added to mannan-coated wells to allow binding of MBL complexes. After wash, human complement C4 was added and incubated at 37° C. The wells were washed and a mixture of two biotin-labeled mAbs against human C4 was added followed by Eu³⁺-labeled streptavidin and measurement of bound Eu³⁺. Results were expressed relative to a standard curve obtained by applying dilutions of a standard serum (10). In separate experiments the amounts of bound MAp44, MAp19 and MASP-2 were measured, as described above.

### Homologies and Phylogenetics

We searched the eukaryote databases for sequences with homologies to human MAp44 and assembled a phylogenetic tree. The 1143 nucleotide long coding sequence (CDS) of the human MAp44 mRNA was compared with sequences in the non-redundant nucleotide database at NCBI using BLASTN (11), identifying full-length similar sequences in Macaca fascicularis (gi:90081135), Mus musculus (gi:26089441), and Rattus norvegicus (gi:55249661). The amino acid sequence of human MAp44 was also blasted against the non-redundant protein database at NCBI using BLASTP with default settings, yielding hits for the translated sequences in the aforementioned animals (Macaca fascicularis, gi:90081136; Mus musculus, gi:148665253 and Rattus norvegicus, gi:55249662), as well as identifying a similar truncated form in Cyprinus carpio, however lacking the 17 aa MAp44 signature (gi:4996234). Genomic alignments and orthologue predictions for the human MASP1 gene were performed using Ensembl (release 50, (12)), identifying homologous transcripts in Pan troglodytes (ENSPTRT00000029309), Macaca mulatta (ENSMMUT00000018241), Canis familiaris (ENSCAFT00000022006) and Danio rerio (ENSDART00000099500). We further identified the protein named MASP-related protein (MRP) from Cyprinus carpio (13), as a MAp44-like protein, as well as an orthologous transcript in Ciona intestinalis (14, 15). The two CDSs of the MASP1/3 gene sequence from Branchiostoma bekheri (32) were compared with the available Branchiostoma floridae genome (JGI, Branchiostoma floridae v1.0, (16)), identifying two homologous regions. In both cases, the exons encoding CCP1 and CCP2 were closely positioned, leaving no space in the intron for an extra MAp44-specific exon. In agreement with this, no sequence homologous to MAp44 could be identified, and no Branchiostoma ESTs or ESTs from related species aligned to this small inter-exonic region.

Xenopus laevis mRNA sequences for MASP1/3a gene product MASP(1) (gi:6429054) (17) and MASP3a (gi:26005766), and MASP3b gene product MASP3b (gi:26005768) were obtained from GenBank, and their respective CDSs compared with the draft of the Xenopus tropicalis genome (JGI, Xenopus tropicalis v4.1), identifying only one gene (scaffold_{—}81:2,412,389-2,470,753), which, as it encodes both MASP-1 and -3, we conclude is the MASP1/3 a gene. The absence of a hit for the MASP3b gene may not be due to the absence of this gene in tropicalis, as opposed to laevis, but rather due to the incompleteness of the draft genome of tropi-calis. An intron (“Intron 8”) of MASP1/3a could putatively accommodate a MAp44 specific exon, but in silico gene prediction failed to identify an exon. BLAST alignment of Xenopus laevis ESTs versus the genomic sequence did, however, identify a single EST (gi:17417909) covering part of exon 5, exon 6, 7, 8, and a sequence in “Intron 8”, which we suspect to be a MAp44-specific exon. The EST sequence was translated revealing a 151 aa uninterrupted sequence. The sequence was BLASTed against NCBI's non-redundant protein database, revealing that the first 142 aa coded for a consecutive CUB and CCP domain similar to MASP-1/-3 from various species, whereas the terminal 9 aa had no obvious similarities. This fits with the sequence representing CUB2-CCP1 and the unique C-terminal of a Xenopus MAp44 orthologue. The genomic region encompassing the MAp44 exon was examined, revealing splice features analogous to the human gene. The aforementioned Xenopus laevis EST was compared with NCBI's non-human, non-mouse EST database using megablast, further identifying 4 overlapping ESTs, all from Xenopus tropicalis (gi:59237729, gi:71452476, gi:59217533, gi:59210250).

The Gallus gallus MASP3 gene (18) was accessed at NCBI, and found to have a sufficiently large “chicken Intron 7” (as the A chain of chicken MASP-1 is only made up of nine exons as compared to ten in mammals, the MAp44 specific exon should possibly be found here in chickens) to accommodate an MAp44 specific exon. This “chicken Intron 7” contained two ESTs, one of them spanning exons 6, 7, and 8 (gi:82782786), the other only covering exon 8 (gi:14004006). A MAp44 se-quence was constructed by joining exons 1-5 from the chicken MASP1 gene with the shared exons 6-7 and the unique exon predicted by EST alignment of gi:82782786. Analogously to the human and Xenopus splice features, the exon has the (C)AG consensus splice acceptor sequence, and two potential branch sites, preceded and followed by polypyrimidine stretches and with no downstream AG dinucleotides until the acceptor AG.

To date, no lizard MASP gene has been described, but when we used the sequences of human MASP-1, -3, and MAp44, Xenopus laevis MASP-1, MASP-3a, MASP-3b, and the putative MAp44, and Gallus gallus MASP-3 and the putative MAp44 mRNA sequences to search the Anolis carolinensis genome (Broad Institute AnoCar (1.0)), a putative MASP-1/-3 encoding gene (scaffold_{—}656:284,678-383,614) was identified with no apparent MAp44-specific exon, but a large “Intron 8”. This intron, “Intron 8”, was BLASTed against the EST database, yielding two ESTs: gi:190286270, which was found to encode a part of exon 6, exon 7, 8, and what was suspected to be an MAp44-specific exon, with 3′UTR and partial polyA tail; and gi:190285980, which was found to encode a small part of exon 5, exon 6, 7, 8 and part of the suspected MAp44-specific exon. The genomic region surrounding this MAp44-specific exon was found to contain the required splice motifs. Based on the sequence alignment of chicken MASP-3 and the identified ESTs with the genomic sequence, the full Anolis carolinensis MAp44 mRNA sequence was assembled.

Bos taurus MAp44 was constructed using Model Maker from Bos taurus MASP-3 mRNA (NM_{—}001076968.1) based on the following bovine ESTs supporting the presence of a MAp44 transcript: gi:112231658 (exon 5-9), gi:87278267 (exon 4-9), gi:82984867 (exon 3-9), gi:17893086 (exon 7-9). The 9th exon in Bos taurus was further supported by ESTs: gi:28151761, gi:28152000, gi:45457641, gi:45470175, and gi:87277042.

Based on the identified translated protein sequences and translations of the identified and reconstructed mRNA transcripts, the MAp44 proteins from human and these 12 organisms were aligned using ClustalX v. 2.0.10 (19) with default settings and iteration at each alignment step: Human (Homo sapiens: gi:73623026), chimpanzee (Pan troglodytes: ENSPTRT00000029309), rhesus macaque (Macaca fascicularis: gi:90081136), long-tailed macaque (Macaca mulatta: ENSMMUT00000018241), cow (Bos taurus, assembled as described), dog (Canis familiaris: ENSCAFT00000022006), mouse (Mus musculus: gi:148665253), rat (Rattus norvegicus: gi:55249662), chicken (Gallus gallus, assembled as described), lizard (Anolis carolinensis, assembled as described), African clawed frog (Xenopus laevis, assembled as described), zebrafish (Danio rerio: ENSDART00000099500), carp (Cyprinus carpio: gi:4996234) and sea squirt (Ciona intestinalis: gi:198422634).

Based on this alignment a consensus bootstrapped N-J tree was produced, excluding positions with gaps and omitting correction for multiple substitutions. The tree was rooted in FigTree v. 1.2.1 using Ciona intestinalis as outgroup (FIG. 7). Presence of the characteristic domain-structure (CUB-EGF-CUB-CCP-tail) in all assembled and retrieved sequences was verified using Swiss-Prot.

### REFERENCES USED IN THE SECTION “EXAMPLES” AND “MATERIALS AND METHODS”

1. Tabaska, J. E., and M. Q. Zhang. 1999. Detection of polyadenylation signals in human DNA sequences. Gene 231:77-86.
2. Liu, H., H. Han, J. Li, and L. Wong. 2005. DNAFSMiner: a web-based software toolbox to recognize two types of functional sites in DNA sequences. Bioinformatics 21:671-673.
3. Blechingberg, J., I. E. Holm, K. B. Nielsen, T. H. Jensen, A. L. Jorgensen, and A. L. Nielsen. 2007. Identification and characterization of GFAPkappa, a novel glial fibrillary acidic protein isoform. Glia 55:497-507.
4. Jensenius, J. C., P. H. Jensen, K. McGuire, J. L. Larsen, and S. Thiel. 2003. Recombinant mannan-binding lectin (MBL) for therapy. Biochem Soc Trans 31:763-767.
5. Thiel, S., S. V. Petersen, T. Vorup-Jensen, M. Matsushita, T. Fujita, C. M. Stover, W. J. Schwaeble, and J. C. Jensenius. 2000. Interaction of C1q and mannan-binding lectin (MBL) with C1r, C1s, MBL-associated serine proteases 1 and 2, and the MBL-associated protein MAp19. J Immunol 165:878-887.
6. Møller-Kristensen, M., J. C. Jensenius, L. Jensen, N. Thielens, V. Rossi, G. Arlaud, and S. Thiel. 2003. Levels of mannan-binding lectin-associated serine protease-2 in healthy individuals. Journal of immunological methods 282:159-167.
7. Teillet, F., C. Gaboriaud, M. Lacroix, L. Martin, G. J. Arlaud, and N. M. Thielens. 2008. Crystal structure of the CUB1-EGF-CUB2 domain of human MASP-1/3 and identification of its interaction sites with mannan-binding lectin and ficolins. J Biol Chem 283:25715-25724.
8. Cseh, S., L. Vera, M. Matsushita, T. Fujita, G. J. Arlaud, and N. M. Thielens. 2002. Characterization of the interaction between L-ficolin/p35 and mannan-binding lectin-associated serine proteases-1 and -2. J Immunol 169:5735-5743.
9. Vorup-Jensen, T., S. V. Petersen, A. G. Hansen, K. Poulsen, W. Schwaeble, R. B. Sim, K. B. Reid, S. J. Davis, S. Thiel, and J. C. Jensenius. 2000. Distinct pathways of mannan-binding lectin (MBL)- and C1-complex autoactivation revealed by reconstitution of MBL with recombinant MBL-associated serine prote-ase-2. J Immunol 165:2093-2100.
10. Petersen, S. V., S. Thiel, L. Jensen, R. Steffensen, and J. C. Jensenius. 2001. An assay for the mannan-binding lectin pathway of complement activation. Journal of immunological methods 257:107-116.
11. Zhang, Z., S. Schwartz, L. Wagner, and W. Miller. 2000. A greedy algorithm for aligning DNA sequences. J Comput Biol 7:203-214.
12. Flicek, P., B. L. Aken, K. Beal, B. Ballester, M. Caccamo, Y. Chen, L. Clarke, G. Coates, F. Cunningham, T. Cutts, T. Down, S. C. Dyer, T. Eyre, S. Fitzgerald, J. Fernandez-Banet, S. Gräf, S. Haider, M. Hammond, R. Holland, K. L. Howe, K. Howe, N. Johnson, A. Jenkinson, A. Kähäri, D. Keefe, F. Kokocinski, E. Kulesha, D. Lawson, I. Longden, K. Megy, P. Meidl, B. Overduin, A. Parker, B. Pritchard, A. Prlic, S. Rice, D. Rios, M. Schuster, I. Sealy, G. Slater, D. Smedley, G. Spudich, S. Trevanion, A. J. Vilella, J. Vogel, S. White, M. Wood, E. Birney, T. Cox, V. Curwen, R. Durbin, X. M. Fernandez-Suarez, J. Herrero, T. J. Hubbard, A. Kasprzyk, G. Proctor, J. Smith, A. Ureta-Vidal, and S. Searle. 2008. Ensembl 2008. Nucleic Acids Res 36:D707-714.
13. Nagai, T., J. Mutsuro, M. Kimura, Y. Kato, K. Fujiki, T. Yano, and M. Nakao. 2000. A novel truncated isoform of the mannose-binding lectin-associated serine protease (MASP) from the common carp (Cyprinus carpio). Immunogenetics 51:193-200.
14. Azumi, K., R. De Santis, A. De Tomaso, I. Rigoutsos, F. Yoshizaki, M. R. Pinto, R. Marino, K. Shida, M. Ikeda, M. Ikeda, M. Arai, Y. Inoue, T. Shimizu, N. Satoh, D. S. Rokhsar, L. Du Pasquier, M. Kasahara, M. Satake, and M. Nonaka. 2003. Genomic analysis of immunity in a Urochordate and the emergence of the vertebrate immune system: “waiting for Godot”. Immunogenetics 55:570-581.
15. Nonaka, M., and F. Yoshizaki. 2004. Primitive complement system of invertebrates. Immunol Rev 198:203-215.
16. Endo, Y., M. Nonaka, H. Saiga, Y. Kakinuma, A. Matsushita, M. Takahashi, M. Matsushita, and T. Fujita. 2003. Origin of mannose-binding lectin-associated serine protease (MASP)-1 and MASP-3 involved in the lectin comple-ment pathway traced back to the invertebrate, amphioxus. J Immunol 170:4701-4707.
17. Putnam, N. H., T. Butts, D. E. Ferrier, R. F. Furlong, U. Hellsten, T. Kawashima, M. Robinson-Rechavi, E. Shoguchi, A. Terry, J. K. Yu, E. L. Benito-Gutiérrez, I. Dubchak, J. Garcia-Fernàndez, J. J. Gibson-Brown, I. V. Grigoriev, A. C. Horton, P. J. de Jong, J. Jurka, V. V. Kapitonov, Y. Kohara, Y. Kuroki, E. Lindquist, S. Lucas, K. Osoegawa, L. A. Pennacchio, A. A. Salamov, Y. Satou, T. Sauka-Spengler, J. Schmutz, T. Shin-I, A. Toyoda, M. Bronner-Fraser, A. Fujiyama, L. Z. Holland, P. W. Holland, N. Satoh, and D. S. Rokhsar. 2008. The amphioxus genome and the evolution of the chordate karyotype. Nature 453:1064-1071.
18. Endo, Y., M. Takahashi, M. Nakao, H. Saiga, H. Sekine, M. Matsushita, M. Nonaka, and T. Fujita. 1998. Two lineages of mannose-binding lectin-associated serine protease (MASP) in vertebrates. J Immunol 161:4924-4930.
19. Lynch, N. J., S. U. Khan, C. M. Stover, S. M. Sandrini, D. Marston, J. S. Presanis, and W. J. Schwaeble. 2005. Composition of the lectin pathway of complement in Gallus gallus: absence of mannan-binding lectin-associated serine protease-1 in birds. J Immunol 174:4998-5006.

### SEQUENCES IN THE APPLICATION

SEQ ID NO: l. MAp44 polypeptide chain starting with the N-terminal
end.
MRWLLLYYALCFSLSKASAHTVELNNMFGQIQSPGYPDSYPSDSEVTWNITVPDG
FRIKLYFMHFNLESSYLCEYDYVKVETEDQVLATFCGRETTDTEQTPGQEVVLSPG
SFMSITFRSDFSNEERFTGFDAHYMAVDVDECKEREDEELSCDHYCHNYIGGYYC
SCRFGYILHTDNRTCRVECSDNLFTQRTGVITSPDFPNPYPKSSECLYTIELEEGFM
VNLQFEDIFDIEDHPEVPCPYDYIKIKVGPKVLGPFCGEKAPEPISTQSHSVLILFHS
DNSGENRGWRLSYRAAGNECPELQPPVHGKIEPSQAKYFFKDQVLVSCDTGYKV
LKDNVEMDTFQIECLKDGTWSNKIPTCKKNEIDLESELKSEQVTE
SEQ ID NO: 2. MAp44 nucleotide sequence
GAAGTCAGCCACACAGGATAAAGGAGGGAAGGGAAGGAGCAGATCTTTTCGGTAGGAAGACAGATTTT
GTTGTCAGGTTCCTGGGAGTGCAAGAGCAAGTCAAAGGAGAGAGAGAGGAGAGAGGAAAAGCCAGAGG
GAGAGAGGGGGAGAGGGGATCTGTTGCAGGCAGGGGAAGGCGTGACCTGAATGGAGAATGCCAGCCAA
TTCCAGAGACACACAGGGACCTCAGAACAAAGATAAGGCATCACGGACACCACACCGGGCACGAGCTC
ACAGGCAAGTCAAGCTGGGAGGACCAAGGCCGGGCAGCCGGGAGCACCCAAGGCAGGAAAATGAGGTG
GCTGCTTCTCTATTATGCTCTGTGCTTCTCCCTGTCAAAGGCTTCAGCCCACACCGTGGAGCTAAACA
ATATGTTTGGCCAGATCCAGTCGCCTGGTTATCCAGACTCCTATCCCAGTGATTCAGAGGTGACTTGG
AATATCACTGTCCCAGATGGGTTTCGGATCAAGCTTTACTTCATGCACTTCAACTTGGAATCCTCCTA
CCTTTGTGAATATGACTATGTGAAGGTAGAAACTGAGGACCAGGTGCTGGCAACCTTCTGTGGCAGGG
AGACCACAGACACAGAGCAGACTCCCGGCCAGGAGGTGGTCCTCTCCCCTGGCTCCTTCATGTCCATC
ACTTTCCGGTCAGATTTCTCCAATGAGGAGCGTTTCACAGGCTTTGATGCCCACTACATGGCTGTGGA
TGTGGACGAGTGCAAGGAGAGGGAGGACGAGGAGCTGTCCTGTGACCACTACTGCCACAACTACATTG
GCGGCTACTACTGCTCCTGCCGCTTCGGCTACATCCTCCACACAGACAACAGGACCTGCCGAGTGGAG
TGCAGTGACAACCTCTTCACTCAAAGGACTGGGGTGATCACCAGCCCTGACTTCCCAAACCCTTACCC
CAAGAGCTCTGAATGCCTGTATACCATCGAGCTGGAGGAGGGTTTCATGGTCAACCTGCAGTTTGAGG
ACATATTTGACATTGAGGACCATCCTGAGGTGCCCTGCCCCTATGACTACATCAAGATCAAAGTTGGT
CCAAAAGTTTTGGGGCCTTTCTGTGGAGAGAAAGCCCCAGAACCCATCAGCACCCAGAGCCACAGTGT
CCTGATCCTGTTCCATAGTGACAACTCGGGAGAGAACCGGGGCTGGAGGCTCTCATACAGGGCTGCAG
GAAATGAGTGCCCAGAGCTACAGCCTCCTGTCCATGGGAAAATCGAGCCCTCCCAAGCCAAGTATTTC
TTCAAAGACCAAGTGCTCGTCAGCTGTGACACAGGCTACAAAGTGCTGAAGGATAATGTGGAGATGGA
CACATTCCAGATTGAGTGTCTGAAGGATGGGACGTGGAGTAACAAGATTCCCACCTGTAAAAAAAATG
AAATCGATCTGGAGAGCGAACTCAAGTCAGAGCAAGTGACAGAGTGAATGACGGGACCCCACACAATG
CAGACATCCAGAAATGGATCACTCCCAAGACCCCTGGGGCCCAGAGCTGCACCACCCCTCCCCACCCA
CAACACCCCCGTGCCCCTTTCCATGTGGATTAGAATGGGTGCTGAACAACATGATCTCAGCAGTTGAA
GCTGCTACGTGTGTGAAAGCAAATTCTCCACTTGAGGGTTTGCCCATCATTCAAACACTATTCCAGAA
AATAATGAAAAAAAAATGTGGGATTTATTTTAGCACCTCTGAGTGGACTGTACTTTTCTCAACGGAAA
AAAAAAATGCCCTTGGTCCTTGAGACAAAAGATTTAATATACAACCATGTGGCCTCAGGCTGACCAGA
TCAAAGTGGTTTCTAATCCATTCTACATGTCAAGTTTAAATGAACCAGACTGCCTGTGACTTTATGAA
TCTGAAGGTATTACCTGTTGCTGCTTTCTTAACCACCATGAGTAGGTAAAGCAAATAATAACTCACAG
AGTGTGGATTTTTGAGAAAAAAAAAAAAAAAAAAAAAAAAAAAAAAAAAAAAAAAAAAAAAAAAAAAA
AAAAAAAAAAAAAAAAAAAAAAAAAAAAAAAA
SEQ ID NO 3 - Protein
CTRAYY
SEQ ID NO 4 - Protein
KNEIDLESELKSEQVTE
SEQ ID NO 5 - Protein
MAGGTRAGT,
wherein M = A/C
SEQ ID NO 6 - Protein
YYYYYYYYYYYNYAG,
wherein Y = C/T
SEQ ID NO 7 - Nucleic acid
CCAGAC

What is claimed is:

1. An isolated polypeptide comprising:
(a) the amino acid sequence of SEQ ID NO 1;
(b) an amino acid sequence with at least about 85%, 90%, 95%, 98%, or 99% sequence identity to SEQ. ID. NO. 1, wherein said polypeptide is capable of binding to mannan-binding lectin (MBL), H-ficolin, L-ficolin, or M-ficolin; or
(c) a fragment of SEQ. ID. NO. 1 that is at least 50, 60, 70, 80, 90, or 100 amino acids in length, wherein said fragment is capable of competitively inhibiting MBL-associated serine proteases (MASPs) or the formation of the MBLIMASP or ficolin/MASP complex.

2. The isolated polypeptide of claim 1, further comprising a label or toxin joined thereto.

3. The isolated polypeptide of claim 1, wherein said polypeptide comprises SEQ ID NO 1.

4. The isolated polypeptide of claim 1, wherein said polypeptide comprises an amino acid sequence with at least about 85%, 90%, 95%, 98%, or 99% sequence identity to SEQ. ID. NO. 1 and, wherein said polypeptide is capable of binding to mannan-binding lectin (MBL), H-ficolin, L-ficolin, or M-ficolin.

5. The isolated polypeptide of claim 1, wherein said polypeptide comprises a fragment of SEQ. ID. NO. 1 that is at least 50, 60, 70, 80, 90, or 100 amino acids in length and, wherein said fragment is capable of competitively inhibiting MBL-associated serine proteases (MASPs) or the formation of the MBLIMASP or ficolin/MASP complex.

6. The isolated polypeptide of claim 3, further comprising a label or toxin joined thereto.

7. An isolated nucleic acid that comprises:
(a) a nucleotide sequence that encodes SEQ ID NO 1;
(b) the nucleotide sequence of SEQ ID NO 2;
(c) a fragment of a nucleotide sequence of SEQ ID NO 2 that is at least about 50, 60, 70, 80, 90, 100, 125, 150, or 200 nucleotides in length; or
(d) a nucleotide sequence with at least about 85%, 90%, 95%, 98%, or 99% sequence identity to SEQ. ID. NO. 2, wherein the polypeptide encoded by said nucleic acid is capable of binding to mannan-binding lectin (MBL), H-ficolin, L-ficolin, or M-ficolin.

8. The isolated nucleic acid of claim 7, wherein said nucleic acid comprises a nucleotide sequence that encodes SEQ ID NO 1.

9. The isolated nucleic acid of claim 7, wherein said nucleic acid comprises the nucleotide sequence of SEQ ID NO 2.

10. The isolated nucleic acid of claim 7, wherein said nucleic acid comprises a fragment of a nucleotide sequence of SEQ ID NO 2 that is at least about 50, 60, 70, 80, 90, 100, 125, 150, or 200 nucleotides in length.

11. The isolated nucleic acid of claim 7, wherein said nucleic acid comprises a nucleotide sequence with at least about 85%, 90%, 95%, 98%, or 99% sequence identity to SEQ. ID. NO. 2 and, wherein the polypeptide encoded by said nucleic acid is capable of binding to mannan-binding lectin (MBL), H-ficolin, L-ficolin, or M-ficolin.

12. The isolated nucleic acid of claim 7, further comprising a vector sequence.

13. The isolated nucleic acid of claim 8, further comprising a vector sequence.

14. The isolated nucleic acid of claim 9, further comprising a vector sequence.

15. The isolated nucleic acid of claim 12, wherein said vector is an expression vector.

16. The isolated nucleic acid of claim 7, further comprising a regulatory element linked operably thereto.

17. The isolated nucleic acid of claim 8, further comprising a regulatory element linked operably thereto.

18. The isolated nucleic acid of claim 9, further comprising a regulatory element linked operably thereto.

19. A method for identifying the presence of MAP44 in a biological sample, comprising
detecting the amount of a polypeptide comprising SEQ ID NO 1 or the amount of an RNA encoding SEQ ID NO 1 in a biological sample.

20. The method of claim 19, further comprising comparing the amount of the polypeptide comprising SEQ ID NO 1 or the amount of the RNA encoding SEQ ID NO 1 detected with a reference amount of MAP44 or an RNA encoding MAP44 or the amount of the polypeptide comprising SEQ ID NO 1 or the amount of the RNA encoding SEQ ID NO 1 detected in a second biological sample.


SEQUENCE LISTING (7 sequences)

SEQ ID NO: 1 - protein, 380 aa, Artificial Sequence
  A MANNAN binding lectin associated Protein MAP 44
MRWLLLYYALCFSLSKASAHTVELNNMFGQIQSPGYPDSYPSDSEVTWNITVPDGFRIKLYFMHFNLESSYLCEYDYVKVETEDQVLATFCGRETTDTEQTPGQEVVLSPGSFMSITFRSDFSNEERFTGFDAHYMAVDVDECKEREDEELSCDHYCHNYIGGYYCSCRFGYILHTDNRTCRVECSDNLFTQRTGVITSPDFPNPYPKSSECLYTIELEEGFMVNLQFEDIFDIEDHPEVPCPYDYIKIKVGPKVLGPFCGEKAPEPISTQSHSVLILFHSDNSGENRGWRLSYRAAGNECPELQPPVHGKIEPSQAKYFFKDQVLVSCDTGYKVLKDNVEMDTFQIECLKDGTWSNKIPTCKKNEIDLESELKSEQVTE

SEQ ID NO: 2 - DNA, 2072 bp, Artificial Sequence
  A MANNAN binding lectin associated Protein MAP 44 encoding nucelic acid
gaagtcagccacacaggataaaggagggaagggaaggagcagatcttttcggtaggaagacagattttgttgtcaggttcctgggagtgcaagagcaagtcaaaggagagagagaggagagaggaaaagccagagggagagagggggagaggggatctgttgcaggcaggggaaggcgtgacctgaatggagaatgccagccaattccagagacacacagggacctcagaacaaagataaggcatcacggacaccacaccgggcacgagctcacaggcaagtcaagctgggaggaccaaggccgggcagccgggagcacccaaggcaggaaaatgaggtggctgcttctctattatgctctgtgcttctccctgtcaaaggcttcagcccacaccgtggagctaaacaatatgtttggccagatccagtcgcctggttatccagactcctatcccagtgattcagaggtgacttggaatatcactgtcccagatgggtttcggatcaagctttacttcatgcacttcaacttggaatcctcctacctttgtgaatatgactatgtgaaggtagaaactgaggaccaggtgctggcaaccttctgtggcagggagaccacagacacagagcagactcccggccaggaggtggtcctctcccctggctccttcatgtccatcactttccggtcagatttctccaatgaggagcgtttcacaggctttgatgcccactacatggctgtggatgtggacgagtgcaaggagagggaggacgaggagctgtcctgtgaccactactgccacaactacattggcggctactactgctcctgccgcttcggctacatcctccacacagacaacaggacctgccgagtggagtgcagtgacaacctcttcactcaaaggactggggtgatcaccagccctgacttcccaaacccttaccccaagagctctgaatgcctgtataccatcgagctggaggagggtttcatggtcaacctgcagtttgaggacatatttgacattgaggaccatcctgaggtgccctgcccctatgactacatcaagatcaaagttggtccaaaagttttggggcctttctgtggagagaaagccccagaacccatcagcacccagagccacagtgtcctgatcctgttccatagtgacaactcgggagagaaccggggctggaggctctcatacagggctgcaggaaatgagtgcccagagctacagcctcctgtccatgggaaaatcgagccctcccaagccaagtatttcttcaaagaccaagtgctcgtcagctgtgacacaggctacaaagtgctgaaggataatgtggagatggacacattccagattgagtgtctgaaggatgggacgtggagtaacaagattcccacctgtaaaaaaaatgaaatcgatctggagagcgaactcaagtcagagcaagtgacagagtgaatgacgggaccccacacaatgcagacatccagaaatggatcactcccaagacccctggggcccagagctgcaccacccctccccacccacaacacccccgtgcccctttccatgtggattagaatgggtgctgaacaacatgatctcagcagttgaagctgctacgtgtgtgaaagcaaattctccacttgagggtttgcccatcattcaaacactattccagaaaataatgaaaaaaaaatgtgggatttattttagcacctctgagtggactgtacttttctcaacggaaaaaaaaaatgcccttggtccttgagacaaaagatttaatatacaaccatgtggcctcaggctgaccagatcaaagtggtttctaatccattctacatgtcaagtttaaatgaaccagactgcctgtgactttatgaatctgaaggtattacctgttgctgctttcttaaccaccatgagtaggtaaagcaaataataactcacagagtgtggatttttgagaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 6 aa, Artificial Sequence
  A predicted optimal branch site consensus sequence of MAP 44
CTRAYY

SEQ ID NO: 4 - protein, 17 aa, Artificial Sequence
  Unique amino acids of MAP 44
KNEIDLESELKSEQVTE

SEQ ID NO: 5 - protein, 9 aa, Artificial Sequence
  MASP 1 consensus sequence
XAGGTXAGT

SEQ ID NO: 6 - protein, 15 aa, Artificial Sequence
  MASP 3 Consensus Sequence
XXXXXXXXXXXNYAG

SEQ ID NO: 7 - DNA, 6 bp, Artificial Sequence
  MAP 44 signal sequence encoding amino acid
ccagac